(19) United States
(12) Patent Application Publication
(10) Pub. No.: US 2026/0167683 A1
(43) Pub. Date: Jun. 18, 2026

(54) CHIMERIC PROTEIN AND EXPRESSION SYSTEM

(71) Applicant: ENDOCRYNE LIMITED, Edinburgh (GB)

(72) Inventor: Ronan Egan, Motherwell (GB)

(21) Appl. No.: 18/697,991

(22) PCT Filed: Oct. 4, 2022
(86) PCT No.: PCT/GB2022/052514

§ 371 Date: Apr. 2, 2024

(30) Foreign Application Priority Data
Oct. 4, 2021 (GB) GB2612021
Oct. 4, 2022 (WO) PCT/GB2022/052514

(51) Int. Cl. C07K 14/46; C07K 14/435; C12N 9/10; C12N 15/62; C12N 15/63

(52) U.S. Cl. CPC C07K 14/46; C07K 14/43518; C12N 9/13; C12N 15/62; C12N 15/63; C12Y 208/01008; C07K 2319/00

(57) ABSTRACT

There is described a novel chimeric protein which reduces oxidative stress in a host cell, which comprises a Bol3 polypeptide operably linked to a Lip5 polypeptide. The chimeric protein can include a linker between the Bol3 polypeptide and the Lip5 polypeptide. The linker can be a polyhistidine linker. Also described is a polynucleotide encoding the chimeric protein, a vector incorporating the polynucleotide, and a host cell transformed with the vector.

A method of expressing a target polypeptide having at least one disulfide bond (for example at least three disulfide e bonds, for example a target polypeptide having an ICK) within a eukaryotic host cell, said method comprising transforming said host cell with a polynucleotide encoding the chimeric protein and culturing said host cell under conditions wherein said chimeric protein and said polypeptide are both expressed.

### TECHNICAL FIELD

The present invention relates to chimeric protein and to a eukaryotic expression system using that chimeric protein for the production of complex disulfide-bonded polypeptides. The expression system is especially useful for the heterologous expression of ‘complex’ post-translationally modified protein products, namely disulfide-rich proteins. Co-expression of the chimeric protein with the complex protein of interest augments cellular fitness to greatly alleviate (‘rescue’) deleterious effects associated with their expression.

The chimeric protein is particularly of utility when the host cell is also expressing a target polypeptide having at least one disulfide bond. Co-expression of the novel fusion protein has been shown to increase the replication (i.e., growth rate) of the yeast and/or the yield of the target polypeptide having at least one disulfide bond.

### BACKGROUND TO THE INVENTION

Polypeptides containing a disulfide-bonded secondary structure typically demonstrate greatly increased chemical, thermal and enzymatic (e.g., resistance to proteolytic digestion) stability, which aids in the bioactivity (i.e., longer half-life and target affinity) of the molecule (Hayward et al., 2017, Journal of Biological Chemistry, 292 (38), 15670-15680; Sermadiras et al., 2013, PLOS ONE, 8 (12), 1-11).

A particular example is venom-derived peptides which typically contain a complex disulfide-rich (3+ bonds) structure, collectively termed an inhibitor cystine knot (“ICK”) motif. The beneficial stability and bioactivity traits observed due to the ICK motif has led to numerous attempts to recombinantly express polypeptides having an ICK motif, to exploit them as novel therapeutic agents (Cao et al., 2003, Peptides, 24 (2), 187-192; Schmoldt et al., 2005, Protein Expression and Purification, 39 (1), 82-89; Sermadiras et al., 2013, supra; Zhong et al., 2014, PLOS ONE, 9 (10), 2-7). This is of note, as in many cases ICK peptides are only present in minute quantities (for example, within the venom secretions), which renders their study and industrial scale-up extremely difficult, costly, and unpredictable (Sermadiras et al., 2013, supra).

Whilst numerous studies have demonstrated that polypeptides comprising an ICK motif can be successfully produced within both bacterial and eukaryotic systems (Sermadiras et al., 2013, supra), expression within a eukaryotic host cell is less successful. The budding yeast, Saccharomyces cerevisiae (S. cerevisiae) is a well-studied and genetically tractable eukaryotic microorganism with a long and proven track record in industrial biotechnology. As with other eukaryotes, disulfide bond formation in yeast takes place within the endoplasmic reticulum (ER) via the concerted action of a 58 kDa protein disulfide isomerase (PDI) and its cognate partner, thiol oxidase Ero1 (65 kDa). To catalyse bond formation, PDI first removes an electron from a cysteine thiol on the target protein, this electron is then shuttled, via Ero1, to a final acceptor, which is typically oxygen (Frand & Kaiser, 1998; Tyo et al., 2012). This shuttle also produces the oxidant, hydrogen peroxide (H₂O₂) in stoichiometric quantities to each disulfide bond produced (Tyo et al., 2012, supra). In addition to this, the yeast's proteostasis machinery, the unfolded protein response (UPR), which maintains and ensures ‘proper’ protein folding, can be activated under these high folding demands, resulting in a further metabolic cost and impact on host fitness (Karagöz, et al., 2019 Cold Spring Harbor perspectives in biology vol. 11, 9).

Consequently, whilst they are attractive bioactive targets for the biotechnology industry, commercial production of recombinant disulfide-bonded proteins requires new strategies to alleviate the metabolic burden (e.g., oxidative stress) incurred upon the host cell.

As a direct result of the metabolic stress-induced through the production of disulfide bonds, expression of such polypeptides yields challenges which are particularly exacerbated where the polypeptide has multiple disulfide bonds. For heterologous expression of ‘complex’ disulfide-containing peptides, such as those which contain an ICK motif, these stresses (e.g., oxidative stresses) can culminate in a number of deleterious outcomes which range from poor host growth metrics (e.g., growth rates, doubling times, etc), exponentially increasing process times (and expenditure), through to lower final product quality, bulk biomass (wet cell weight, g/L), and yield.

In terms of product quality, unbridled oxidant production also increases the likelihood of adduct formation via protein oxidation, specifically carbonylation, which can adversely affect the quality of the final product (Yang, et al., 2014 Analytical Chemistry, 86 (10), 4799-4806). A protein adduct is a covalent modification resulting from reactions between electrophiles and nucleophilic sites in proteins, such as at the N-terminus or at an amino acid side chain containing sulfhydryl or amine functionalities. The addition of carbonyl groups to a protein is an example of an adduct.

The present invention addresses such problems. In particular, the present invention provides a chimeric protein (or “chimera”) which significantly alleviates the poor growth (growth rate, generations per hour) of transgenic host cells expressing a target polypeptide having at least one disulfide bond, for example, a target polypeptide comprising an ICK motif.

The present invention also provides a method of expressing a target polypeptide having at least one disulfide bond, for example, a target polypeptide comprising an ICK motif, which leads to improved host cell fitness and/or to improved target polypeptide yield. An expression system for the production of a target polypeptide having at least one disulfide bond, for example a target polypeptide comprising an ICK motif, is also described.

### SUMMARY OF THE INVENTION

The present invention provides a novel chimeric protein that comprises a Bol3 polypeptide operably linked to a Lipoyl synthase (“Lip5”) polypeptide. The chimeric protein can include a linker between the Bol3 polypeptide and the Lip5 polypeptide. The linker can conveniently allow flexibility and/or can facilitate separation of the Bol3 domain from the Lip5 domain in the chimeric protein.

Additionally, the present invention provides a polynucleotide encoding the chimeric protein, a vector incorporating the polynucleotide, and a host cell transformed with the vector.

In a further aspect, the present invention provides a method of expressing a target polypeptide having at least one disulfide bond (for example, a target polypeptide having at least three disulfide bonds, for example a target polypeptide having at least three disulfide bonds in the form of an ICK motif) within a eukaryotic host cell, said method comprising transforming said host cell with a polynucleotide encoding the chimeric protein and culturing said host cell under conditions wherein said chimeric protein and said target polypeptide are expressed. Notably, in addition expression of the chimeric protein itself has been found to be well tolerated by the host as its expression alone does not negatively impact cellular growth rates.

The present invention further provides an expression system for expression of a target protein of interest, the system comprising an expression vector comprising the chimeric protein according to the invention and a cloning site for insertion of a polynucleotide encoding the target polypeptide of interest. Typically, the target polypeptide will have at least one disulfide bond (for example, a target polypeptide having at least three disulfide bonds, for example a target polypeptide having at least three disulfide bonds in the form of an ICK motif).

### BRIEF DESCRIPTION OF THE FIGURES

FIG. 1. A: TAE gels of amplification; and B: schematic of OE-PCR.

FIG. 2. Plasmid map illustration. Multiple cloning sites (MCS) 1 and 2 illustrated, MCS-1 contains the chimeric open reading frame.

FIG. 3. Boxplot of growth rates between a chimeric protein according to the invention (Chimera) and control strain (Control). Data demonstrates no significant difference (i.e., no loss of fitness) when yeast expresses Chimera. N=12, one-way ANOVA used for significance, no significance indicated by ‘NS’.

FIG. 4. Purification of chimeric protein according to the invention. A single band at the approximate molecular weight of the chimeric protein resolved in Fraction-3 and 4. 12% SDS-PAGE gel, 20 μL loading volume with 5 μL of PageRuler Prestained Protein ladder.

FIG. 5. Boxplot of growth rates between a chimeric protein according to the invention (Chimera-1) and control strain (Control) under oxidative stress. Data demonstrates that expression of the chimera facilitates resistance to up to 5 mM hydrogen peroxide. N=6 per condition, one-way ANOVA used for significance. **=p<0.01, NS.=no significance.

FIG. 6. Boxplot of growth rates between a chimeric protein according to the invention (Chimera) and control strain (Control) under reductive stress. N=6 per condition, one-way ANOVA used for significance. ***=p<0.001, n.s.=no significance.

FIG. 7. A: Gel images of evasin gene (SEQ ID No. 19) (EVA); and B: polypeptide product (SEQ ID No. 18) (EVA) purified by NiNTA affinity chromatography.

FIG. 8. A: Multiple sequence comparison and structure of C8 evasins. Eight evasin variants demonstrating the (8) conserved cysteine residues. B: Structure of the C8 evasin family, illustrating the cystine knot (ICK motif).

FIG. 9. Boxplot demonstrating maximum growth rates of evasin-expressing yeast (‘Evasin-2’) and its rescue by coexpression of chimera (Chi; +Evasin-2). One-way ANOVA was used for significance. ***=p<0.001, NS.=no significance.

FIG. 10. Schematic of peptides, indicating the location of the cystine knots present in each peptide. Cystines labelled with ‘C’ followed by location in primary sequence.

FIG. 11. Competitive lateral flow assay for polyhistidine-tagged polypeptides, Purotoxin-1, Psalmotoxin-1 and Evasin-2. Band pattern indicates successful expression of desired products.

FIG. 12. Boxplot demonstrating the effect of other ICK polypeptides (Purotoxin-1, Psalmotoxin-1) on the growth rates of yeast (S. cerevisiae). One-way ANOVA was used for significance. ***=p<0.001, *=p<0.05, NS.=no significance.

FIG. 13. Fermentation of Chimera;+EVA. Batch mode, results (wet cell weight in g/L, final OD₆₀₀ and hours to dissolved oxygen setpoint) of each batch.

FIG. 14. Fermentation of EVA (Evasin-2). Batch mode, results (wet cell weight in g/L, final OD₆₀₀ and hours to dissolved oxygen setpoint) of each batch.

FIG. 15. Fermentation of Purotoxin-1 co-expressing Chimera. Batch mode, results (wet cell weight in g/L, final OD₆₀₀ and hours to dissolved oxygen setpoint) of each batch.

FIG. 16. Fermentation of Purotoxin-1. Batch mode, results (wet cell weight in g/L, final OD₆₀₀ and hours to dissolved oxygen setpoint) of each batch.

### DETAILED DESCRIPTION OF THE INVENTION

The chimeric protein, polynucleotides and vectors encoding the chimeric protein, expression system and methods of the present invention are now described in further detail.

As used herein, the term “and/or” is to be taken as specific disclosure of each of the two specified features or components with or without the other.

As used herein, the term “comprising” is to be construed as encompassing both “including” and “consisting of”, both meanings being specifically intended, and hence individually disclosed embodiments in accordance with the present invention.

As used herein the term “polypeptide” refers to a polymer composed of amino acids joined by peptide bonds and does not refer to a specific length of the polymer. A “peptide bond” is a covalent bond between two amino acids in which the α-amino group of one amino acid is bonded to the α-carboxyl group of the other amino acid. The polypeptide can be modified, for example by glycosylation, amidation, carboxylation, phosphorylation, or the like. The modification can be in vitro or in vivo. Amino acid chains with a length of less than approximately 100 amino acids are generally considered within the art to be “peptides”, but both “peptides”, and “proteins” are included within the definition of “polypeptides” as used herein. The terms “amino acid sequence” and “polypeptide sequence” are used interchangeably. All amino acid or polypeptide sequences, unless otherwise designated, are written from the amino terminus (N-terminus) to the carboxy terminus (C-terminus).

For convenience of nomenclature, this application refers to a “chimeric protein” (or “chimera”) and “a target polypeptide having at least one disulfide bond”. However, the designation of “protein” in the term “chimeric protein” and of “polypeptide” in the term “a target polypeptide having at least one disulfide bond” is not intended to suggest any information regarding the size or relative size of the two polymers concerned.

The present invention is particularly concerned with the expression of a target polypeptide having at least one disulfide bond. Disulfide bonds are formed by the covalent bonding of the thiol groups of two cysteine residues within the polypeptide. Two cysteine residues are required for each disulfide bond. As explained above, the formation of the disulfide bond leads to oxidative stress in the host cell. Optionally, the target polypeptide has two or more disulfide bonds. Optionally, the target protein has three or more disulfide bonds. Optionally, the target protein having at least one disulfide bond has an ICK, as defined further below. Optionally, the target polypeptide can include another cystine motif, such as a cyclic cystine knot or a Growth Factor cystine knot, or the like.

An “inhibitor cystine knot” or “ICK” refers to a motif within a polypeptide comprising at least 3 pairs of cysteine residues which form three separate disulfide bonds. Two disulfide bonds form a loop through which the third disulfide bond (linking the 3^{rd} and 6^{th} cysteine in the sequence) passes, forming a knot.

As used herein, when applied to an amino acid sequence, “conservative substitution” refers to the substitution of one amino acid residue with another amino acid residue having a side chain with similar physical and chemical properties. For example, conservative substitution may be conducted among amino acid residues having a hydrophobic side chain (e.g., Met, Ala, Val, Leu, and Ile), amino acid residues having a neutral hydrophilic side chain (e.g., Cys, Ser, Thr, Asn, and Gln), amino acid residues having an acidic side chain (e.g., Asp and Glu), amino acid residues having a basic side chain (e.g., His, Lys, and Arg), or amino acid residues having an aromatic side chain (e.g., Trp, Tyr and Phe). It is known in the art that a conservative substitution generally does not cause a significant change in the conformational structure of a protein, and thus can retain the biological activity of the protein.

The term “polynucleotide” refers to a polymer of nucleic acid, for example, DNA, cDNA, RNA or synthetically produced DNA or RNA or a recombinantly produced chimeric polynucleotide molecule comprising one of these polynucleotides alone or in combination. The term “nucleic acid” is used interchangeably with the term “polynucleotide”.

The term “vector” as used herein refers to a genetic construct to facilitate the handling of a target polynucleotide. The vector may comprise further genes such as marker genes, which allow for the selection of the vector in a suitable host cell and under suitable conditions. Expression of said polynucleotide or vector comprises transcription of the polynucleotide into a translatable mRNA. Usually, a vector comprises regulatory sequences ensuring initiation of transcription. Other elements which are responsible for the initiation of transcription, such as regulatory elements, may also be present. The vector may also comprise transcription termination signals downstream of the target polynucleotide.

When applied to an amino acid sequence (or a nucleic acid sequence), “percent sequence identity” refers to a percentage of amino acid (or nucleic acid) residues in a candidate sequence that are identical to those of a reference sequence, relative to the amino acid (or nucleic acid) residues in the candidate sequence during sequence alignment, and if necessary, after introducing gaps to maximize the number of identical amino acids (or nucleic acids). A conservative substitution of amino acid residue may or may not be considered as an identical residue. Percent sequence identity of amino acid (or nucleic acid) sequences can be determined by aligning sequences through tools disclosed in the art. A person skilled in the art may use the default parameters of the tools or adjust the parameters appropriately according to the needs of the alignment, for example by choosing an appropriate algorithm. The percentage identity between two polypeptide sequences may be readily determined by programs such as BLASTp which is freely available at http://blast.ncbi.nlm.nih.gov.

An “isolated” material has been artificially altered from its natural state. If an “isolated” substance or component occurs in nature, it has been altered or removed from its original state, or both. For example, a polynucleotide or polypeptide naturally occurring in a living animal is not isolated but may be considered “isolated” if the polynucleotide or polypeptide is sufficiently isolated from the materials with which it coexists in its native state and exists in a sufficiently pure state. In some embodiments, the polynucleotide or polypeptide are at least 90%, 93%, 95%, 96%, 97%, 98%, 99% pure as determined by electrophoresis (e.g., SDS-PAGE, isoelectric focusing, capillary electrophoresis), or chromatography (e.g., ion-exchange chromatography or reverse phase HPLC).

The terms “variant”, “homologue” or “derivative” in relation to a nucleotide sequence include any substitution of, variation of, modification of, replacement of, deletion of or addition of one (or more) nucleic acid(s) from or to the sequence.

In a first aspect, the present invention provides a novel chimeric protein which comprises a Bol3 polypeptide operably linked to a Lip5 polypeptide. The chimeric protein can include a linker between the Bol3 polypeptide and the Lip5 polypeptide. Expression of the chimeric protein reduces oxidative stress in a host cell and finds particular utility when the host is also expressing a target polypeptide having at least one disulfide bond.

The term “Bol3” is used to reference the Bol3 protein of yeast (for example S. cerevisiae) but is also used herein to refer to the homologues of this protein in other species, in particular to homologues of the Bol3 protein in eukaryotes (such as the Bol3A homologue in mice, bovine and human cells) and E. coli.

In one embodiment the Bol3 polypeptide comprises at least 50% sequence identity to SEQ ID NO: 1.

Optionally, the Bol3 polypeptide has more than 50% sequence identity to SEQ ID NO: 1, for example has at least 55%, 60%, 65% 70%, 75% 80%, 85% or 90% sequence identity to SEQ ID NO: 1. Optionally, Bol3 polypeptide has more than 90% sequence identity to SEQ ID NO: 1, for example has 95% or more, for example has 98% or more, sequence identity to SEQ ID NO: 1. SEQ ID NO: 1 is the sequence of the Bol3 protein in S. cerevisiae.

Optionally, the Bol3 polypeptide has more than 50% sequence identity to a protein expressed from SEQ ID NO: 4, for example has at least 55%, 60%, 65% 70%, 75% 80%, 85% or 90% sequence identity to a protein expressed from SEQ ID NO: 4. Optionally, the Bol3 polypeptide has more than 90% sequence identity to a protein expressed from SEQ ID NO: 4, for example has 95% or more, for example has 98% or more, sequence identity to a protein expressed from SEQ ID NO: 4. SEQ ID NO: 4 is a polynucleotide sequence encoding the Bol3 protein in S. cerevisiae without the native stop codon, as used in the chimeric protein described in the examples.

The term “Lip5” is used to reference the Lip5 protein of yeast (for example S. cerevisiae), but is also used herein to refer to the homologues of this protein in other species, in particular to homologues of the Lip5 protein in eukaryotes, in plants and in E. coli.

Optionally, the Lip5 polypeptide comprises at least 50% sequence identity to SEQ ID NO: 2. Optionally, the Lip5 polypeptide has more than 55% sequence identity to SEQ ID NO: 2, for example has 60%, 65%, 70%, 75%, 80%, 85% or 90% sequence identity to SEQ ID NO: 2. Optionally, the Lip5 polypeptide has more than 90% sequence identity to SEQ ID NO: 2, for example has 95% or more, for example has 98% or more, sequence identity to SEQ ID NO: 2.

Optionally, the Lip5 polypeptide comprises at least 50% sequence identity to a protein expressed from SEQ ID NO: 5. Optionally, the Lip5 polypeptide has more than 55% sequence identity to a protein expressed from SEQ ID NO: 5, for example has 60%, 65%, 70%, 75%, 80%, 85% or 90% sequence identity to a protein expressed from SEQ ID NO: 5. Optionally, the Lip5 polypeptide has more than 90% sequence identity to a protein expressed from SEQ ID NO: 5, for example has 95% or more, for example has 98% or more, sequence identity to a protein expressed from SEQ ID NO: 5. SEQ ID NO: 5 is a polynucleotide sequence encoding the Lip5 protein in S. cerevisiae without the native start codon, as used in the chimeric protein described in the examples.

In one embodiment a linker sequence is located between the Bol3 polypeptide and the Lip5 polypeptide. The term “linker” as used herein describes a group or sequence that allows the two portions of the chimeric protein to be linked. For example, the linker allows the Bol3 polypeptide and the Lip5 polypeptide to be linked together. The linker serves to connect the two components. The linker according to the present invention can be flexible or rigid, but more preferably allows some flexibility between the Bol3 and Lip5 portions of the chimeric protein. Suitable linkers are known to the skilled person. More specifically, the term “linker” refers to a peptide chain consisting of 1-50 amino acids forming a peptide bond, or a derivative thereof, the N- and C-termini of which form a covalent bond with either the Bol3 domain or the Lip5 domain, respectively, thereby binding the Bol3 domain to the Lip5 domain.

Optionally, the linker sequence is a polyhistidine linker. For example, the linker sequence can include from 6 to 20 (for example 8 to 16, for example 8 to 12) histidine residues in a polyhistidine linker, that is the linker comprises from 6 to 20 consecutive histidine residues to form a polyhistidine linker.

Other suitable linkers are known in the art and include FLAG tag, Cys tag, GST tag and the like. Another suitable linker is the N-terminal portion of the Cia2 protein (see SEQ ID No. 28), optionally with additional linking amino acids. SEQ ID No: 28 shows the N-terminal portion of the Cia2 protein, and optionally the linker can comprise further amino acids, for example the sequence of SEQ ID NO: 29 can be used as a linker. Optionally, a polynucleotide sequence encoding this the N-terminal portion of the Cia2 protein can be used with additional nucleotides to ensure in-frame cloning. For example, SEQ ID No: 30 shows a polynucleotide encoding a suitable linker sequence, with the Cia2 sequence being encoded by nucleotides 19 to 52 inclusive. SEQ ID No: 29 shows the amino acid sequence encoded by SEQ ID No: 30. Thus, the linker can be a sequence comprising the sequence of SEQ ID NO: 29.

One embodiment of the invention is a chimeric protein comprising a first amino acid sequence of Bol3 having at least 50% sequence identity to SEQ ID NO: 1 or at least 50% sequence identity to a polypeptide encoded by SEQ ID NO: 4, a linker peptide and a second amino acid sequence of Lip5 having at least 50% sequence identity to SEQ ID NO: 2 or at least 50% sequence identity to a polypeptide encoded by SEQ ID NO: 5. Optionally, the linker sequence is a polyhistidine linker.

For example, the linker sequence can include from 6 to 20 (for example 8 to 16, for example 8 to 12) histidine residues in a polyhistidine linker. For example, the linker sequence can be the N-terminal portion of the Cia2 protein. Optionally the linker can comprise or consist of the sequence of SEQ ID NO: 28 or of SEQ ID No: 29.

Optionally, the sequence identity of the Bol3 polypeptide in the chimeric protein to SEQ ID NO: 1 or to a polypeptide encoded by SEQ ID NO: 4 in the chimeric protein described above is greater than 50%, for example is at least 55%, 60%, 65% 70%, 75% 80%, 85% or 90%. Optionally, the Bol3 polypeptide in the chimeric protein described above has more than 90% sequence identity to SEQ ID NO: 1 or to a polypeptide encoded by SEQ ID NO: 4.

Optionally, the sequence identity of the Lip5 polypeptide in the chimeric protein to SEQ ID NO: 2 or to a polypeptide encoded by SEQ ID NO: 5 in the chimeric protein described above is greater than 50%, for example is at least 55%, 60%, 65% 70%, 75% 80%, 85% or 90%. Optionally, the Lip5 polypeptide in the chimeric protein described above has more than 90% sequence identity to SEQ ID NO: 2 or to a polypeptide encoded by SEQ ID NO: 5.

One embodiment of the invention is a chimeric protein comprising a first amino acid sequence of Bol3 having at least 95% sequence identity to SEQ ID NO: 1 or to a polypeptide encoded by SEQ ID NO: 4, a linker peptide and a second amino acid sequence of Lip5 having at least 95% sequence identity to SEQ ID NO: 2 or to a polypeptide encoded by SEQ ID NO: 5. Optionally, the linker sequence is a polyhistidine linker. For example, the linker sequence can include from 6 to 20 (for example 8 to 16, for example 8 to 12) histidine residues in a polyhistidine linker. Optionally the linker sequence is the N-terminal portion of the Cia2 protein (see SEQ ID NO: 28). Optionally the linker can comprise or consist of the sequence of SEQ ID NO: 28 or of SEQ ID No: 29.

In some embodiments, the first amino acid sequence (Bol3) has at least 96%, 97%, 98%, 99% or more sequence identity to the amino acid sequence of SEQ ID NO: 1 or to a polypeptide encoded by SEQ ID NO: 4.

In some embodiments, the second amino acid sequence (Lip5) has at least 96%, 97%, 98%, 99% or more sequence identity to the amino acid sequence of SEQ ID NO: 2 or to a polypeptide encoded by SEQ ID NO: 5.

Optionally, the chimeric protein comprises at least 70% sequence identity to the amino acid sequence of SEQ ID NO: 3, for example has at least 75%, 80%, 85% 90%, or 95% sequence identity to the amino acid sequence of SEQ ID NO: 3. Optionally, the chimeric protein has at least 91%, 92%, 93%, 94%, 95%, 96%, 97%, 98%, 99% or more sequence identity to the amino acid sequence of SEQ ID NO: 3.

In one embodiment of the present invention, the chimeric protein comprises the amino acid sequence shown in SEQ ID NO: 3.

In one embodiment of the invention, the chimeric protein has at least 50% sequence identity to a protein expressed from SEQ ID NO: 31. Optionally, the chimeric protein has more than 55% sequence identity to a protein expressed from SEQ ID NO: 31, for example has 60%, 65%, 70%, 75%, 80%, 85% or 90% sequence identity to a protein expressed from SEQ ID NO: 31. Optionally, the chimeric protein has more than 90% sequence identity to a protein expressed from SEQ ID NO: 31, for example has 95% or more, for example has 98% or more, sequence identity to a protein expressed from SEQ ID NO: 31. Optionally, the chimeric protein is encoded by SEQ ID NO: 31.

In a second aspect of the invention, the invention provides a polynucleotide which encodes the chimeric protein described above. In addition, the invention also encompasses a polynucleotide which specifically hybridizes under stringent conditions to the polynucleotide encoding the chimeric protein. For the purposes of the present specification, hybridisation under stringent hybridisation conditions means remaining hybridised after washing with 0.1×SSC, 0.5% SDS at a temperature of at least 68° C., as described by Sambrook et al (Molecular Cloning. A Laboratory Manual. Cold Spring Harbor Press).

Optionally, the invention provides an isolated polynucleotide. According to an embodiment of the present invention, the isolated polynucleotide encodes the chimeric protein as described above. Thus, the isolated polynucleotide according to the present invention can be used to encode a chimeric protein which reduces oxidative stress within the host cell.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed.

The polynucleotide of the invention may consist of DNA or RNA. The polynucleotide may be single-stranded or double-stranded. The polynucleotide may include synthetic or modified nucleotides. Several different types of modification to polynucleotides are known in the art. These include methylphosphonate and phosphorothioate backbones, addition of acridine or polylysine chains at the 3′ and/or 5′ ends of the molecule. For the purposes of the invention as described herein, it is to be understood that the polynucleotides may be modified by any method available in the art. Such modifications may be carried out to enhance the in vivo activity or life span of polynucleotides of interest.

Optionally, the polynucleotide of the invention comprises a sequence having at least 50% sequence identity to the nucleotide sequence of SEQ ID NO: 4. Optionally, the polynucleotide of the invention comprises a sequence having at least 55%, 60%, 65%, 70%, 75%, 80%, 85% or 90% sequence identity to the nucleotide sequence of SEQ ID NO: 4. Optionally, the polynucleotide of the invention comprises at least 91%, 92%, 93%, 94%, 95%, 96%, 97%, 98%, 99% or more than 99% sequence identity to the nucleotide sequence of SEQ ID NO: 4.

Optionally, the polynucleotide of the invention comprises a sequence having at least 50% sequence identity to the nucleotide sequence of SEQ ID NO: 5.

Optionally, the polynucleotide of the invention comprises a sequence having at least 55%, 60%, 65%, 70%, 75%, 80%, 85% or 90% sequence identity to the nucleotide sequence of SEQ ID NO: 5. Optionally, the polynucleotide of the invention comprises at least 91%, 92%, 93%, 94%, 95%, 96%, 97%, 98%, 99% or more than 99% sequence identity to the nucleotide sequence of SEQ ID NO: 5.

Optionally, the polynucleotide of the invention has a nucleotide sequence which expresses a chimeric protein with at least 70% sequence identity to SEQ ID NO: 3. Optionally, the polynucleotide of the invention encodes a polypeptide having a sequence identity to SEQ ID NO. 3 which is more than 70%, for example which is 75%, 80%, 85%, 90% or even more. Optionally, the polynucleotide of the invention encodes a polypeptide having at least 91%, 92%, 93%, 94%, 95%, 96%, 97%, 98%, 99% or more than 99% sequence identity to the nucleotide sequence of SEQ ID NO: 3.

Optionally the polynucleotide of the invention has at least 50% sequence identity to the nucleotide sequence of SEQ ID NO: 32. Optionally, the polynucleotide of the invention comprises a sequence having at least 55%, 60%, 65%, 70%, 75%, 80%, 85% or 90% sequence identity to the nucleotide sequence of SEQ ID NO: 32. Optionally, the polynucleotide of the invention comprises at least 91%, 92%, 93%, 94%, 95%, 96%, 97%, 98%, 99% or more than 99% sequence identity to the nucleotide sequence of SEQ ID NO: 32.

Optionally the polynucleotide of the invention encodes a polypeptide which comprises the nucleotide sequence of SEQ ID NO: 3.

Optionally the polynucleotide of the invention encodes a polypeptide which comprises the nucleotide sequence of SEQ ID NO: 31.

In a third aspect, the present invention provides a vector comprising such a polynucleotide, in particular an expression vector expressing, or overexpressing, said polynucleotide.

A “vector” in the present invention refers to a vehicle into which a polynucleotide encoding a protein can be operably inserted for enabling the protein to be expressed. The vector can be used to transform, transduce, or transfect (which terms are used interchangeably herein) a host cell, such that the genetic elements carried by the vector are expressed in the host cell. A variety of vectors are available. The vector may comprise a variety of elements that control expression, including a promoter sequence, a transcription initiation sequence, an enhancer sequence, a signal sequence, one or more marker genes, a selection element, a reporter gene, and a transcription termination sequence. Further, the vector may also comprise an origin of replication. The vector may also comprise a component that facilitates the vector to enter into cells, including, but not limited to, viral particle, liposome, or protein shell.

For example, the vectors include plasmids, phagemids, cosmids, artificial chromosomes such as yeast artificial chromosome (YAC), bacterial artificial chromosome (BAC) or P1-derived artificial chromosome (PAC), bacteriophages such as 2 bacteriophage or M13 bacteriophage, animal viruses, and the like.

In some embodiments, the vector systems include mammalian, bacterial, and yeast systems, and will include plasmids such as, but not limited to, ‘pENDO-2’ and other vectors available from the laboratory or commercially available vectors. Suitable eukaryotic vectors include vectors having a 2 micron or centromeric origin of replication. Suitable vectors may include plasmid or viral vectors (e.g., replication-defective retroviruses, adenoviruses, and adeno-associated viruses).

The present invention thus provides an expression vector comprising the above polynucleotide. The expression vector of the present embodiment can be prepared by subcloning the polynucleotide as described above into the expression vector by any conventionally known genetic engineering method. The type of expression vector that can be used in the present embodiment is not particularly limited, and examples thereof include any expression vector suitable for heterologous gene expression in eukaryotes and able to drive expression of the target polypeptide. For example, a eukaryotic vector having a 2 micron or centromeric origin of replication together with a constitutive promoter/terminator cassette can conveniently be used, for example the Tef1 promoter.

A vector comprising a polynucleotide encoding the chimeric protein may be introduced into a host cell for cloning (amplification of DNA) or gene expression using recombinant techniques well known in the art. In another embodiment, the chimeric protein can be prepared by homologous recombination methods well known in the art.

Thus, in a fourth aspect, the present invention provides a host cell comprises a vector or a polynucleotide as described above.

A “host cell” in the present invention refers to a cell into which an exogenous polynucleotide and/or a vector are introduced. Amino acid sequences of the fusion protein of the present application may be converted to corresponding DNA coding sequences using genetic engineering techniques well known in the art. Due to the degeneracy of genetic code, the transformed DNA sequences may not be completely identical, while the encoded protein sequences remain unchanged.

Host cells suitable for cloning or expressing the DNA in the vectors of the present invention are prokaryotic, yeast or the above-mentioned advanced eukaryotic cells. Prokaryotic cells suitable for use in the present invention include E. coli (for example E. coli DH5α and BL21de3).

In one embodiment, eukaryotic host cells are used for cloning or expressing vectors encoding the chimeric protein. Saccharomyces cerevisiae (S288C) or baker's yeast is the most used lower eukaryotic host microorganism. However, many other genera, species and strains are common and suitable for use in the present invention, such as other members of the Saccharomyces clade (including S. pastorianus. S. eubayanus and S. paradoxus), Komagataella (including K. pastoris), Kluyveromyces (including K. lactis) and Yarrowia (including Y. lipolytica). In another aspect of the present invention, the present invention provides a recombinant cell or recombinant microorganism which contains the polynucleotide or vector as described above. Thus, the recombinant cells or recombinant microorganisms according to the present invention can express the chimeric protein of the present invention. The invention further relates to a recombinant host cell comprising the polynucleotide, or the vector as described above. The polynucleotide or vector of the present invention, which is present in the host cell, may either be integrated into the genome of the host cell, or it may be maintained extra-chromosomally. Once the polynucleotide or vector has been incorporated into the appropriate “host cell”, the host cell is maintained under conditions suitable for high level expression of the polynucleotide or vector.

The transformed host cells can be grown according to methodology known in the art to achieve cell growth.

Optionally, once expressed, the chimeric protein can be purified according to standard procedures of the art. Mention may be made of affinity columns, column chromatography, such as size exclusion chromatography (SEC), gel electrophoresis, ammonium sulphate precipitation and the like. The chimeric protein of the invention can then be isolated from the growth medium, cellular lysates, or cellular membrane fractions. The isolation and purification of the chimeric protein may be by any conventional means such as, for example, preparative chromatographic separations.

The host cell is transformed with the above-mentioned expression or cloning vector that can produce the chimeric protein, and then cultured in a conventional nutrient medium, which is suitable for inducing promoters, selecting transformed cells, or amplifying genes encoding target sequences after being modified.

The host cells used to produce the chimeric protein in the present invention can be cultured in a variety of media known in the art. The media may also comprise any other necessary additives known in the art in a suitable concentration. The conditions of the media, such as temperature, pH and the like are those selected previously for expression of host cells, which are well known to those of ordinary skill.

The present invention further provides a method for producing a chimeric protein as described above, wherein the method comprises the following steps of suitably culturing a recombinant host cell comprising and expressing a polynucleotide encoding the chimeric protein or a vector encoding the chimeric protein.

In a fifth aspect, the present invention provides a method for producing a target polypeptide having at least one disulfide bond in a host cell, wherein said method includes: culturing the host cell under conditions suitable for the expression of the chimeric protein together with expression of the target polypeptide.

The target polypeptide can be a naturally occurring or a synthetic polypeptide.

Optionally, the target polypeptide has more than one disulfide bond to create its desired 3D structure, for example has 2, 3, 4 or 5 disulfide bonds. Optionally, the disulfide bonds are formed between non-adjacent cysteine residues, that is, the disulfide bonds form a complex 3D arrangement of “knot” in the target polypeptide. Optionally the target polypeptide includes an “inhibitor cystine knot” or “ICK”.

Optionally the target polypeptide is a venom polypeptide, for example is derived from the “Evasin” family of salivary peptides. Optionally the target polypeptide has at least 90% sequence identity to any one of SEQ ID NOS: 6 to 13. Optionally the target polypeptide has at least 90% sequence identity to SEQ ID NO: 18, SEQ ID NO: 20 or SEQ ID NO: 22.

Optionally the target polypeptide is a venom polypeptide, for example is derived from the “Purotoxin” from the Wolf Spider, Alopecosa marikovskyi (SEQ ID NO: 20, or encoded by SEQ ID NO: 21) and/or ‘Psalmotoxin-1’ (UniProt ID: TXP1_PSACA) from the Trinidad chevron tarantula Psalmopoeus cambridgei, (SEQ ID NO: 22 or encoded by SEQ ID NO: 23). Alternatively, the target polypeptide can be other polypeptides of interest which have a disulfide bond, for example Factors ‘C’ and ‘B’ from the Atlantic Horseshoe crab Limulus polyphemus and/or ‘Coagulogen-1’ (UniProt ID: COAG_TACTR) from Tachypleus tridentatus (Japanese horseshoe crab), or Hemocyanin-1′ (UniProt ID: HCY1_MEGCR) from Megathura crenulata (Giant keyhole limpet) and Hemocyanin-2′ (UniProt ID: HCY2_MEGCR) from Megathura crenulata (Giant keyhole limpet, as well as Bovine serum albumin, BSA, UniProtKB—P02769 (ALBU_BOVIN); Human serum albumin, HSA, UniProt-B—P02768 (ALBU_HUMAN), Human Insulin (including Human Insulin analogues and Human insulin mimetic peptides) UniProtKB—P01308 (INS_HUMAN), Human Erythropoietin UniProtKB—P01588 (EPO_HUMAN) or Human Granulocyte-macrophage colony-stimulating factor UniProtKB—P04141 (CSF2_HUMAN).

Optionally, the target polypeptide can be an antibody, for example a monoclonal antibody, a humanised antibody or an antibody fragment.

Optionally, the target polypeptide can be a glycoprotein, for example a glycoprotein with a secretion sequence (i.e., a glycoprotein which is secreted from the host cell).

The host cell can be genetically engineered to express said target polypeptide. For example, the host cell can be transformed with an expression vector comprising a polynucleotide sequence encoding the target polypeptide. The expression vector may either be integrated into the genome of the host cell, or it may be maintained extra-chromosomally. Once the vector has been incorporated into the appropriate “host cell”, the host cell is maintained under conditions suitable for high-level expression of the target polypeptide.

The host cell can be genetically engineered to express the target polypeptide and then further genetically engineered to express the chimeric protein, or vice versa. Optionally the host cell naturally expresses the target polypeptide and is simply transformed to express the chimeric protein as described above.

Optionally a single vector comprising polynucleotides encoding both the chimeric protein and also the target polypeptide can be formed, and the host cell is then simply transformed with the vector able to express both the target polypeptide and also the chimeric protein. Optionally both polynucleotides are under the control of the same promoter/inducer/enhancer.

In a further aspect the present invention provides an expression vector comprising a polynucleotide encoding the chimeric protein as described above and a cloning site for insertion of a polynucleotide encoding a target polypeptide. The cloning site may be defined by suitable restriction sites, allowing the easy insertion of the polynucleotide encoding the target polypeptide. For example, the expression vector may include a multiple cloning site, having up to 20 different restriction sites to facilitate easy insertion of different constructs for the target polypeptide.

In a yet further aspect, the present invention provides an expression system for expressing a target polypeptide of interest in a host cell, said system comprising an expression vector comprising a polynucleotide encoding the chimeric protein as described above and vector comprising a cloning site for insertion of a polynucleotide encoding a target polypeptide. Optionally, the cloning site may be provided on the vector encoding the chimeric protein of the invention. Alternatively, a separate vector can be provided with a cloning site for expression of the target polypeptide. The cloning site may be defined by suitable restriction sites, allowing the easy insertion of the polynucleotide encoding the target polypeptide. For example, the expression vector may include multiple cloning sites, having up to 20 different restriction sites to facilitate easy insertion of different constructs for the target polypeptide.

The target polypeptide can be a naturally occurring or a synthetic polypeptide.

Optionally, the target polypeptide has at least one disulfide bond, and may include more than one disulfide bond to create its desired 3D structure, for example has 2, 3, 4 or 5 disulfide bonds. Optionally, the disulfide bonds are formed between non-adjacent cysteine residues, that is, the disulfide bonds form a complexed 3D arrangement of “knot” in the target polypeptide. Optionally the target polypeptide includes an “inhibitor cystine knot” or “ICK”.

Optionally the target polypeptide is a venom polypeptide, for example is derived from the “Evasin” family of salivary peptides. Optionally the target polypeptide has at least 90% sequence identity to any one of SEQ ID NOS: 6 to 13. Optionally the target polypeptide has at least 90% sequence identity to SEQ ID NO: 18, SEQ ID NO: 20 or SEQ ID NO: 22.

Optionally the target polypeptide is a venom polypeptide, for example is derived from the “Purotoxin” from the Wolf Spider, Alopecosa marikovskyi (see SEQ ID NOS: 20 and 21) and/or ‘Psalmotoxin-1’ (UniProt ID: TXP1_PSACA) from the Trinidad chevron tarantula Psalmopoeus cambridgei (see SEQ ID NOS: 22 and 23). Optionally, the target polypeptide can be Factor ‘C’ or ‘B’ from the Atlantic Horseshoe crab Limulus polyphemus; ‘Coagulogen-1’ (UniProt ID: COAG_TACTR) from Tachypleus tridentatus (Japanese horseshoe crab), or Hemocyanin-1′ (UniProt ID: HCY1_MEGCR) from Megathura crenulata (Giant keyhole limpet) and Hemocyanin-2′ (UniProt ID: HCY2_MEGCR) from Megathura crenulata (Giant keyhole limpet; Bovine serum albumin (BSA, UniProtKB—P02769 (ALBU_BOVIN); Human serum albumin (has) UniProt-B—P02768 (ALBU_HUMAN), Human Insulin UniProtKB—P01308 (INS_HUMAN), Human Erythropoietin UniProtKB—P01588 (EPO_HUMAN) or Human Granulocyte-macrophage colony-stimulating factor UniProtKB—P04141 (CSF2_HUMAN).

Optionally, the target polypeptide can be an antibody, for example a monoclonal antibody, a humanised antibody or an antibody fragment.

Optionally, the target polypeptide can be a glycoprotein, for example a glycoprotein with a secretion sequence (i.e., a glycoprotein which is secreted from the host cell).

Optionally, once expressed, the target polypeptide can be purified according to standard procedures of the art. Mention may be made of affinity columns, column chromatography, such as size exclusion chromatography (SEC), gel electrophoresis, ammonium sulphate precipitation and the like. The target polypeptide can then be isolated from the growth medium, cellular lysates, or cellular membrane fractions. The isolation and purification of the target polypeptide may be by any conventional means such as, for example, preparative chromatographic separations.

All documents referred to herein are incorporated by reference. Any modifications and/or variations to described embodiments that would be apparent to one of skill in art are hereby encompassed. Whilst the invention has been described herein with reference to certain specific embodiments and examples, it should be understood that the invention is not intended to be unduly limited to these specific embodiments or examples.

Preferred or alternative features of each aspect or embodiment of the invention apply mutatis mutandis to each other aspect or embodiment of the invention (unless the context demands otherwise).

### EXAMPLES

### Methods

### Strains, Culture Conditions and Materials

Oligonucleotides and sequences for this study were designed first using in silico cloning software, with reference from the Saccharomyces genome database and then purchased from ThermoFisher custom oligo ordering service. Templates for PCR were prepared from fresh overnight cultures of Saccharomyces cerevisiae (BY4741, MATa his3Δ1 leu2Δ0 met15Δura3Δ0) using 20 mg/mL Lyticase (Sigma Aldrich, UK) digestion at +37° C. in a digital dry block (ThermoFisher, UK) followed by purification by total genomic spin prep kit from New England Biolabs (Monarch, New England Biolabs, UK). For sub-cloning, 10 μL of electrocompetent Escherichia coli (DH5a) cells (New England Biolabs, UK) were routinely used and plasmids selection under positive antibiotic selection 100 μg/mL Ampicillin supplemented in Luria Bertani (LB) media. Transformation of E. coli was performed according to the manufacturer's instructions and transformants were incubated at +37° C., static, for at least 16 hours. Before use, all aliquots and buffers were briefly centrifuged at maximum speed (15,500 r.c.f.) for at least 60 seconds.

### Creation of Fusion ORF

Two methodologies were used for creating the fusion ORF; the first methodology used was adapted from Hilgarth & Lanigan, (2020), MethodsX, 7 (October 2019), 100759. For latter constructs, traditional restriction enzyme cloning was used.

Stage 1: High Fidelity DNA polymerase for POR was used as a (2×) master mix (Hot Start Q5 High Fidelity, NEB UK), containing 4 mM MgCl₂, and 2 mM dNTP mix. Routinely, PCRs were performed at 50 μL in clean, thin walled 0.2 mL tubes (ThermoFisher, UK) and prepared on ice and mixed thoroughly by pulse vortexing at maximum speed (Stuart, UK) Thermocycling was performed in a 24-well Prime³ thermocycler (Techne, UK) for 2 hours and 30 minutes, with a preheated lid (+105° C.). General thermocycling conditions for stage 1 and stage 3 of OE-PCR were 10 seconds of initial denaturing at +98° C., 35 cycles of +98° C. for 10 seconds, 60 seconds of +60° C. and then an extension stage of +72° C. for 2 minutes seconds. This was performed for a total of 35 cycles. Finally, a final extension phase was of minutes at +72° C. was performed. The reaction was then held at +20° C.

After thermocycling, amplicons were briefly reconstituted by pulse centrifugation at 15,000 r.c.f, for approximately 10 seconds. Following this, the reaction was confirmed by TAE gel electrophoresis using 0.7% w/v agarose (FisherSci, UK) and 0.05% v/v EtBr (Sigma-Aldrich, UK), as a DNA intercalator. Gels were run for 30 minutes at 150 V, 400 mA using a small gel tank (Alpha labs, UK) and electrophoresis Powerpack (FisherSci, UK). After finishing, gels were carefully visualised under blue light (proBLUEView, Alpha Labs, UK). To determine approximate molecular weights of the amplicons, 10 μL of GeneRuler 1 kb (ThermoFisher, UK) was used as a standard. The PCR yielded two single bands at approximately 350 bps and 1200 bps which matched the reference molecular weights for Bol3 and Lip5 (Saccharomyces genome database, yeastgenome.org), respectively. Gel fragments were then excised using a clean scalpel and purified by commercial spin-column protocol (GeneJet Gel Extraction Kit, ThermoFisher, UK), according to the manufacturer's instructions.

Stage 2: The second stage used the in-built complementarity between each amplicon to fuse both ORFs. This was fulfilled by the addition of a 30-mer polyhistidine (10×) sequence with a slightly higher melting temperature (+68° C.) than that of the annealing sequences. This region formed a ‘linker’ between each ORF. Codons for histidine were alternated to avoid tRNA depletion. Unlike stage 1, this stage utilised a touch-down PCR protocol to enable a higher degree of sensitivity towards the polyhistidine linker region. Templates for touchdown PCR consisted of an equimolar (1:1) concentration (ng/μL) of amplicons generated in Stage 1. Calculations were performed using a ligation calculator, where the shorter sequence was considered as ‘insert’. PCR was again performed using a preheated lid to mitigate evaporation. Thermocycling consisted of 9 cycles of denaturation at +95° C. for 30 seconds, followed by 3 minutes of annealing at +72° C. for 15 seconds, with a 0.5° C. decrease in annealing temperature per cycle. Subsequently, 5 cycles of denaturing at +95° C. for 30 seconds, followed by annealing at +67.5° C. for 30 seconds, following this a 3 minute 30-second extension step was performed at +68° C. Finally, an extension period was performed at +68° C. for 10 minutes.

Stage 3: The final stage of OE-PCR utilised the un-purified PCR products of stage 2 as a template. Thermocycling was performed under the same programme as stage 1, albeit with different oligonucleotides. Here, oligonucleotides against the 5′ (Forward) and 3′ (Reverse) of the first and second ORFs were used to amplify only fused sequences generated in stage 2. The oligonucleotides used are shown below in Table 1. As before, TAE gel electrophoresis was performed to confirm the success of the fusion reaction. Here, a single band representing the combined molecular weight of both ORF was detected. The gel slice was then excised, and DNA purified via a commercial gel extraction kit, as above.

TABLE 1
Oligonucleotide Primers

| Oligo name | Sequence (5′ - 3′) |
|---|---|
| SEQ ID No: 14 | CACCATCACCATCACCATCACCATCACCATCACC |
| Lip5_F | TTAGGCTTTATAGACGATCTGTTGGAGTACTATT |
| | TGTTGGGAGAAA |
| SEQ ID No: 15 | cccccCCGCGGTTATTATTTCATGTTTCTTTTCT |
| Lip5_R | TCAAAACGTTCTCAATAAATGCTTCAC |
| SEQ ID No: | cccccTCTAGATACACAATGAAGCTCCCACAGAC |
| 16Bol3 F | CATGCTACGTTC |
| SEQ ID No: 17 | ATGGTGATGGTGATGGTGATGGTGATGGTGATGA |
| Bol3_R | GGCCTAAGTGATGATGCCGGACCCTTCCCAGTTG |
| SEQ ID No. 24, | cccccTCTAGATACACAATGAAGCTCCCACAGAC |
| BOL_fv2 | CAT |
| SEQ ID No. 25, | gggggGCGGCCGCAGGCCTAAGTGATGATGCCGG |
| BOL_rv2.1 | ACCCTTC |
| SEQ ID No. 26, | cccccGCGGCCGCTTCTGGGGAGCGGCCTGTGAC |
| Lip5_IDR_F | GGCAGGCGAGGAGGACCTTAGGCTTTATAGACGA |
| | TCTGTTGGAGTACTATTTGTTGG |
| SEQ ID No. 27, | gggggCCGCGGTTATTATTTCATGTTTCTTTTCT |
| LIP5_IDR_R | TCAAAACGTTCTCAATAAATGC |

### Restriction Enzyme Digests and Subsequent Cloning

Restriction enzymes (Xba I, Not I and Sac II) were obtained from the CutSmart range of New England Biolabs (New England Biolabs, UK) and digestions (50 μL) performed in 1× CutSmart buffer according to the manufacturer's instructions, at +37° C. for 2 hours within a digital dry batch. After digestion, any condensate was removed via pulse centrifugation at maximum speed (15,500 r.c.f.) in a benchtop centrifuge (SciQuip, UK). DNA ligations (20 μL) were likewise performed using a Quick Ligation Kit (New England Biolabs, UK) according to the manufacturer's instructions, using a 5:1 molar ratio of insert to vector where 1 ratio of vector was standardised at 27 fmol. DNA (ng/μL) was routinely quantified using a UV/Vis spectrophotometer with spectra (260-700 nm) (SpectroStar Nano, BMG Labtech, UK). Following incubation, 2 μL of the ligation reaction was transformed into electrocompetent DH5α E. coli cells, on ice. After at least 16 hours of incubation at +37° C. (allowing colony growth), individual colonies were analysed for successful ligation via diagnostic digest and colony PCR. Approximately 20 colonies were screened per ligation in a final reaction volume of 20 μL. This protocol repeated Stage 3 as above, albeit using one (marked) colony per reaction. Successful amplification was then be relayed back to the individual colonies for plasmid purification via commercial MiniPrep kits (GeneJet MiniPrep Kit, ThermoFisher). After purification, plasmid eluates were labelled and stored at −20° C.

### Rapid Yeast Transformations

Introduction of the newly created plasmid into S. cerevisiae host was performed using an overnight culture of BY4741 and pre-dried uracil drop out plates (Kaiser minimal drop-out media, Formedium, UK). Before performing the reaction, 1 mL of single-stranded DNA at 1 mg/ml (Ultrapure Salmon sperm, Sigma Aldrich, UK) was boiled at +95° C. for 10 minutes and then immediately placed on ice.

Transformations were performed using a reaction mixture containing 240 μL of 50% w/v polyethylene glycol (PEG₄₀₀₀, Melford, UK), 36 μL of 1 M lithium acetate (Sigma Aldrich, UK), 10 μL of freshly boiled single-stranded carrier DNA, 7.2 μL of 5 M DTT (Melford, UK), 2 μL of plasmid and finally 69.5 μL of sterile milli Q water. All solutions and buffers were sterilised by autoclaving before use.

After assembling the reaction mixture, the mixture was thoroughly vortexed at maximum speed for at least one minute per transformation incubated at room temperature for 20 minutes and then heat shocked at +42° C. for a further 20 minutes. Following this, the reaction mixture was pelleted by slow centrifugation at 2000 r.c.f, for 2 minutes, then gently resuspended in 200 μL of sterile deionised water and plated onto pre-dried drop-out plates. Plates were sealed and colonies appeared after 4 days of incubation at +30° C.

### High-Resolution Growth Rate Analysis

Yeast strains were Incubated overnight (at least 16 hours) at +30° C., 175 r.p.m. in 10 mL of synthetic defined Kaiser dropout media (uracil drop out, Formedium). Following incubation, the density of each culture was quantified by spectroscopy (SpectroStar Nano, BMG Labtech, UK) at an optical density of 600 nanometres (OD_{600 nm}) in a 1 mL cuvette (BMG Labtech, UK). Cultures were then loaded into an OT-2 liquid handling robot (Opentrons, USA) and diluted back to an optical density (OD_{600 nm}) of 0.1 in a sterile, flat-bottomed 96-well plate (360 μL well-volume, Greiner CELLSTAR® 96 well plates, Sigma Aldrich UK). The growth of each strain was then monitored continuously until saturation at which point the experiment was terminated and data collected.

### Batch Fermentations

Fermentations were performed using a culture of recombinant yeast grown overnight in synthetic defined uracil dropout media (Kaiser, Formedium, UK). Routinely, a working volume of 100 mL (250 mL total volume) was used consisting of 0.67 g of Yeast Nitrogen Base without amino acids (Formedium, UK), 0.19 g of relevant amino acid supplement and 2 g of anhydrous D-glucose (Melford, UK). The reactor (MiniBio 250 mL total volume, Applikon Biotechnology, NL) was then assembled, and its contents sterilised by autoclaving in a Prestige Medical Classic Autoclave (+121° C., 104 kPa, 30 minutes). Following this, the reactor was connected to a MiniBio Fermentation control system (Applikon Biotechnology, NL), tubing (alkali, air) connected, and probes (pH and Dissolved oxygen) left to polarise overnight at room temperature. This step also served as a sterility control.

Overnight cultures (5 mL) of the yeast were prepared in relevant drop out media and incubated for 16 hours, 175 r.p.m., at +30° C. as above. Meanwhile, probes were calibrated as follows: dissolved oxygen (DO₂) was calibrated to read 100% DO₂ (approx. 70 nA at +30° C.) in the un-inoculated media. pH was calibrated in (20 mL) standards of pH 4.0 and pH 7.0 (Sigma, UK). The next morning, strains were sub-cultured (5 mL) to an OD_{600 nm} of 0.2 and incubated a second time for 4 hours, 175 r.p.m., at +30° C. After this time had elapsed, the bioreactor was inoculated with a precalculated inoculum volume (mL) to 0.1 (OD_{600 nm}) and Lucullus Process Information Management Software (SecureCell, CH) was used to monitor the fermentation. Setpoints were: 35% DO₂±5%, pH 5.0±0.5 and 1 vvm sparging with compressed air (Bambi PT5 UK). Routinely, total fermentation time was 20 hours.

### Downstream Processing

After fermentation, the total sample (approximately 120 mL, OD_{600 nm} of approximately 35-55, strain dependent) was extracted and decanted into two separate 50 mL falcon tubes (ThermoFisher, UK). 15 mL of culture were then pelleted by centrifugation at 15,500 r.c.f, for 10 minutes, weighed (wet cell weight, g/L) and then lysed in Yeast Protein Extraction Reagent (YPER, Pierce, UK), according to manufacturer's instructions.

### Chemical Cell Lysis (Protein Extraction)

Sample lysis was performed as described for “Downstream processing” above. The cell pellet was in an appropriate volume of YPER (according to manufacturer's Instructions) and agitated at 1800 r.p.m. (Stuart Vortex, UK) for 20 minutes at room temperature with Pierce Protease Inhibitor Tablets (Thermo Scientific, UK). After which the sample was clarified by centrifugation to clear insoluble debris and the supernatant aspirated into a clean 1.5 mL Eppendorf tube (Eppendorf, UK), for further analysis.

### Affinity Purifications

Immobilised metal affinity chromatography (IMAC) was performed to confirm the expression of the fusion protein of 60 kDa. The individual molecular weights of Bol3 and Lip5 polypeptides are 13 and 46 kDa, respectively (source, Saccharomyces Genome database, yeastgenome.org). For purification, 1 mL of HisPur Nickel chromatography resin (Sigma, UK) was aliquoted into an empty PD-10 (10 mL, Sigma-Aldrich, UK) column and equilibrated with 1 column volume of denaturing binding buffer (8 M Urea, 10 mM Imidazole, 137 mM NaCl, 2.7 mM KCl, 10 mM Na₂HPO₄, 1.8 mM KH₂PO₄, 20% v/v Glycerol pH 7.4). To this, the sample prepared in “Chemical cell lysis (Protein Extraction)” was carefully loaded by pipetting and allowed to run through by gravity. The run-through was collected and labelled ‘RT’ for further analysis. Washes were performed using (8 M Urea, 50 mM Imidazole, 137 mM NaCl, 2.7 mM KCl, 10 mM Na₂HPO₄, 1.8 mM KH₂PO₄, 20% v/v Glycerol, pH 7.4). Protein was eluted using elution buffer B, (8 M Urea, 50 mM Imidazole, 137 mM NaCl, 2.7 mM KCl, 10 mM Na₂HPO₄, 1.8 mM KH₂PO₄, 20% v/v Glycerol, pH 7.4) and collected in 15×1 mL fractions (1.5 mL Eppendorf tubes). All samples were maintained on ice for the duration of the purification. UV/Vis spectroscopy (SPECTROstar Nano, UK) at 280 nm using an LVis plate (BMG, UK) was used to quantify protein concentration (mg/mL).

### Detection of Recombinant Proteins by Sodium Dodecyl Sulfate-Polyacrylamide Gel Electrophoresis (SDS-PAGE)

Fractions from the affinity purification step above were analysed by sodium dodecyl sulfate-polyacrylamide gel electrophoresis (SDS-PAGE) at 100 V for 10 minutes and 180 V, 200 mA for 50 minutes. Polyacrylamide gels were prepared according to Laemmli stack method with a 10% v/v resolving gel (Surecast, Resolving buffer pH 8.8, Thermo Fisher, UK) and 4% v/v stacking gel (Surecast Stacking buffer, pH 6.8, Thermo Fisher, UK) with polymerisation induced with 50 μL of 10% w/V ammonium persulphate (Sigma, UK) and 5 μL of 100% v/v tetramethylethylenediamine (TEMED, Melford, UK).

Samples were denatured in 4× Laemmli reducing buffer, diluted to working concentration (1×) in a final volume of 20 μL and then heated to +100° C. for 5 minutes in a thermocycler (Prime3, Techne, UK). Per sample, 20 μL of diluted (1:20) sample was loaded per well alongside 5 μL of PageRuler prestained molecular weight marker (ThermoFisher, UK). After the run gels were removed and stained using SimplyBlue protein stain (Invitrogen, UK) according to manufacturer's instructions. Once stained, gels were recorded using white light transillumination (proBLUE View, Cleaver Scientific).

### Competitive Lateral Flow Assay for Polyhistidine-Tagged Polypeptides

Pro-Detect™ Rapid His Competitive Assay Kit (Thermo Scientific, UK) was used to confirm expression of recombinant polyhistidine-tagged polypeptides according to manufacturer's instructions.

### Data Analysis

Data analysis and graphing were routinely performed in RStudio (RStudio, V4.0.01) and later visualised with Adobe Illustrator CC (2020).

Growth rates were calculated using ‘GrowthCurver’ script available at: https://cran.rproject.org/web/packages/growthcurver/vignettes/Growthcurver-vignette.html. Grammar of graphics (ggplot2) was routinely used for graphic design, available at: https://ggplot2.tidyverse.org/.

Fermentation data was recorded and visualised using Lucullus Process Information Management Software (Applikon, Getinge, NL and SecureCell, CH).

### Results

### Example 1: Episomal Expression of the Recombinant Bol3-Lip5 Chimera

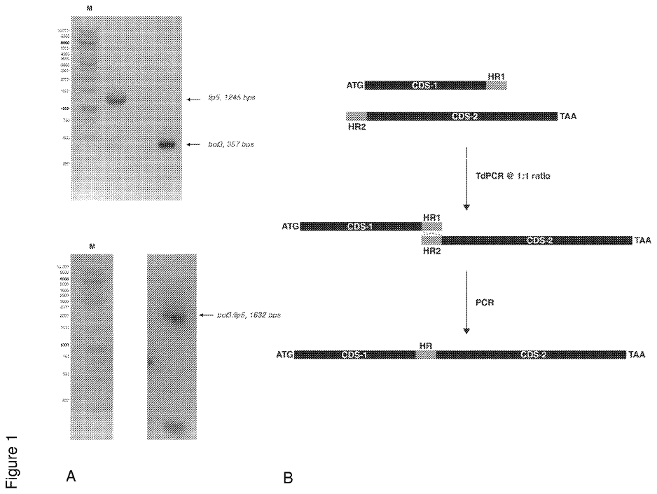

Overlap extension PCR was used to fuse the two open reading frames of bol3 and lip5 into the single fused open reading frames, FIG. 1B, which lacked the native stop and start codons of bol3 and lip5, respectively, in order to render a single fused transcript. When amplified the final Chimeric amplicon migrated to an approximate molecular weight of 1632 bps. This was in agreement with the expected combined weights of both bol3 and lip5, FIG. 1A.

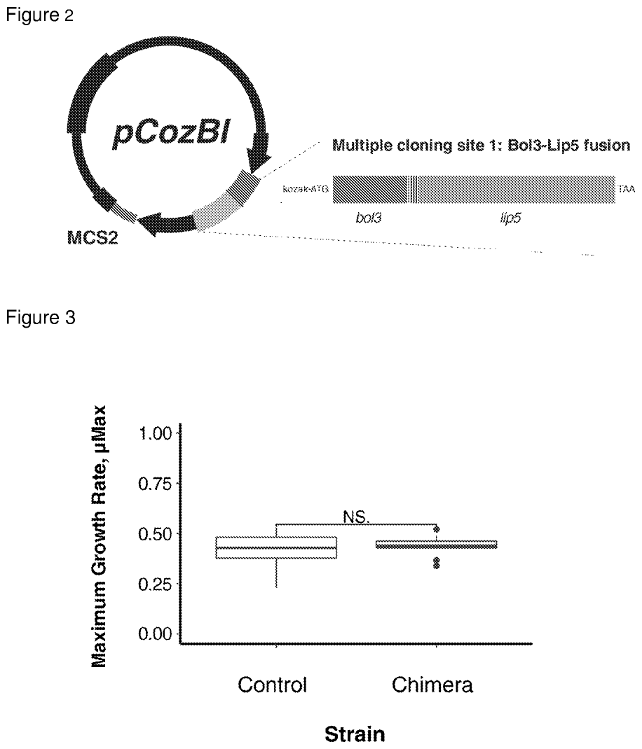

Amplicons were then gel excised, digested, and ligated into a centromeric yeast expression construct before transformation into chemically competent DH5α E. coli, as per the manufacturer's (New England Biolabs, UK) instructions. The resulting construct (FIG. 2) was then transformed into Saccharomyces cerevisiae (BY4741) according to a modified LiAC/PEG method (Gietz, et al., 2002 Methods in Enzymology, Volume 350, Pages 87-96), using 100 mM DTT.

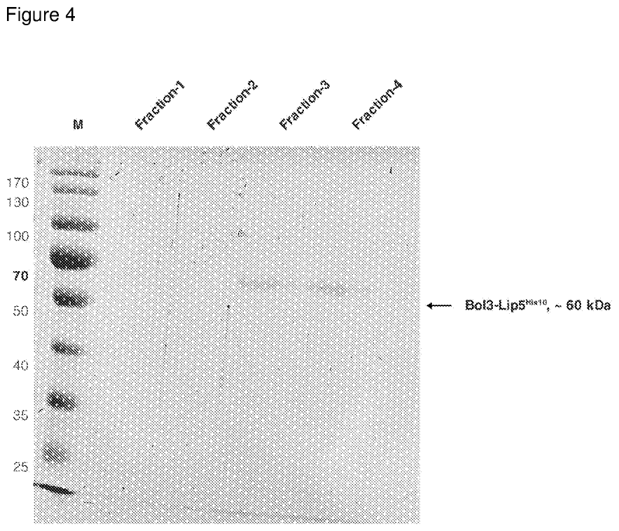

Owing to the presence of the polyhistidine motif which served to link both ORFs, it was possible to purify the fused polypeptide from yeast lysate via nickel column chromatography. The result was a single polypeptide which resolved at an approximate molecular weight of approximately 60 kDa, in agreement with the combined weights of both Bol3 (13 kDa) and Lip5 (46 kDa) polypeptides, FIG. 4.

Expression of the Chimeric Protein does not Adversely Affect Yeast Growth Rate, but does Aid in Relieving Pressure from Environmental Stimuli.

High-resolution growth rate analysis determined that the cellular fitness of the newly generated chimera-expressing yeast strain, was statistically indistinguishable (CI=95%) from controls (FIG. 3). These data suggest that the chimera is well tolerated within the yeast system.

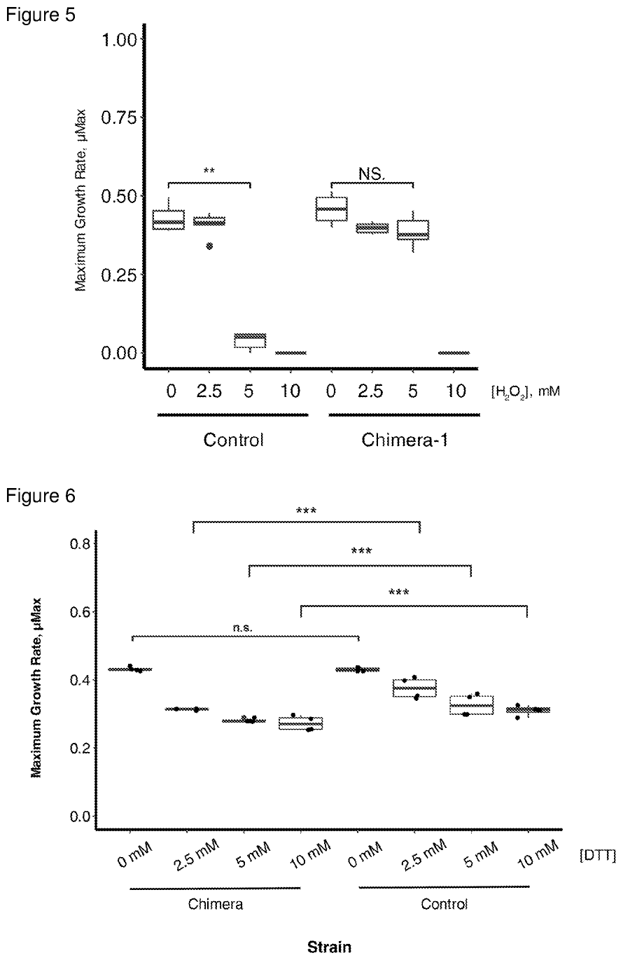

Further experiments under sub- to lethal oxidative conditions (hydrogen peroxide) demonstrated that chimera expression did afford a good degree of protection up to a peroxide concentration of 10 mM, FIG. 5. Importantly, this observation was unique to chimera-expressing yeast.

Countering this, experiments within a reductive setting demonstrated that chimera expression conveyed a heightened sensitivity to the disulfide bond disruptant, dithiothreitol (DTT), compared to controls, FIG. 6.

### Example 2: Expression of the Recombinant Venom-Derived Peptide, EVA

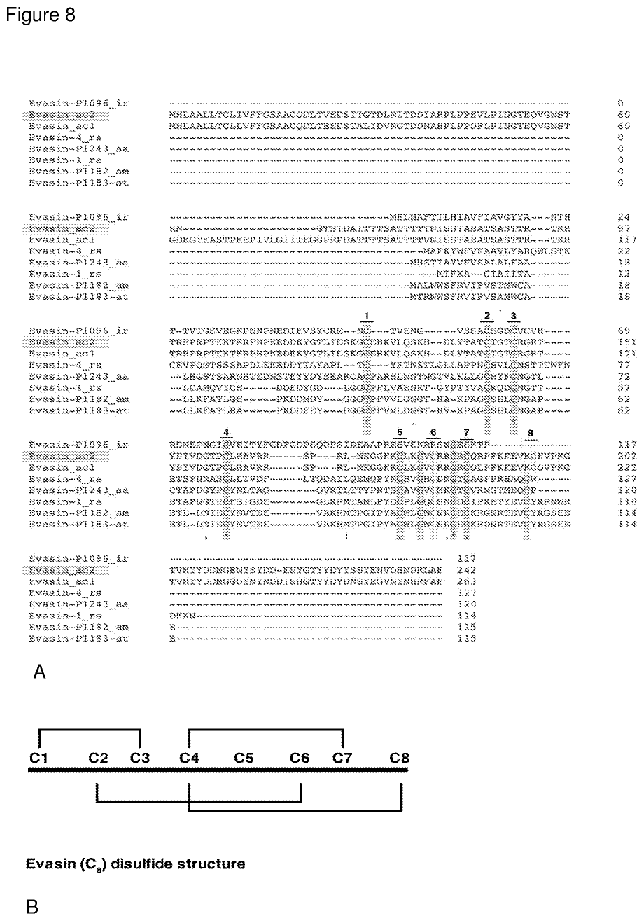

The synthetic polypeptide, ‘EVA’ (SEQ ID No. 18), is derived from a secreted protein of the tick ‘Evasin’ family of bioactive salivary peptides (FIG. 8) (Hayward et al., 2017 supra). Within their host organisms (including Amblyomma cajennense, ‘Cajun tick’), Evasins are secreted by the salivary gland to promote parasite survival by targeting and sequestering CXXC and CXC chemo- and cytokines, which are released by the host to eliminate the parasite (Denisov et al., 2019, Journal of Biological Chemistry, 294 (33), 12370-12379). By sequestering these molecules, the tick effectively dampens the host's defences (immune cell chemotaxis) and thus, prolongs its feeding and parasitic life cycle (Denisov et al., 2019, supra). Recently, this mechanism has drawn attention as a potential therapeutic agent for treating (calming) otherwise fatal ‘cytokine storms’, associated with both viral and other disease states (Darlot et al., 2020, The Journal of Biological Chemistry, 295 (32), 10926-10939).

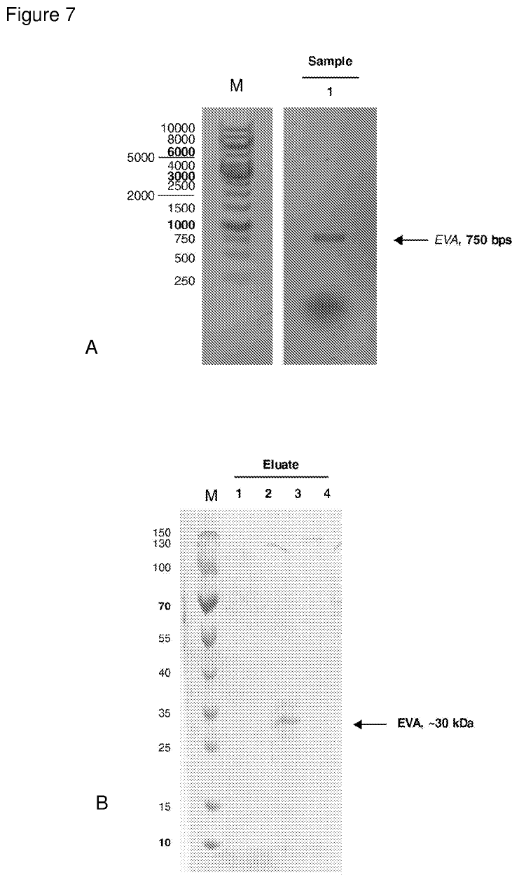

After determining that the yeast cultures were harbouring the recombinant EVA gene (SEQ ID NO: 19), as shown in FIG. 7A, protein expression was confirmed using nickel chromatography and SDS-PAGE. The results of this are shown in FIG. 7B and demonstrate that a dense band was resolved at roughly the equivalent predicted molecular weight of EVA (approximately 30 kDa). An antibody-based assay was used to confirm the presence of polyhistidine-tagged polypeptide within the fraction (FIG. 11).

### Example 3: Yeast Harbouring EVA Exhibit a Severe, Temperature-Dependent Reduction in Growth Rate Compared to Controls

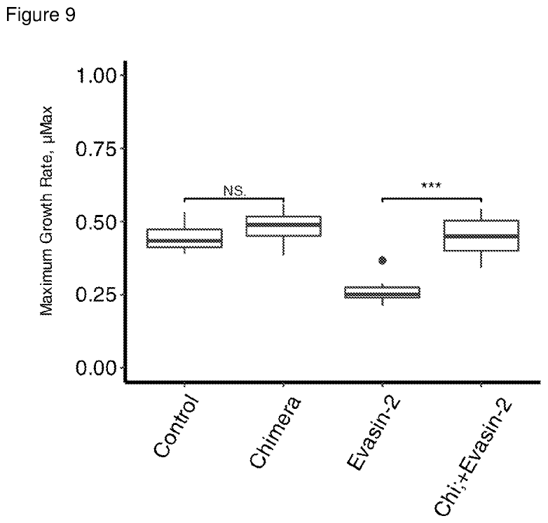

High-resolution growth rate analysis at both +30 and +32 degrees Celsius demonstrated that the expression of EVA conveys a significant (p<0.001) decrease in growth rate compared to controls (wildtype and empty vector) (FIG. 9). These data (summarised in Table 2) demonstrated that expression of EVA results in a roughly 60 and 40% reduction in relative growth rate compared to wildtype and empty vector controls, respectively. This is worsened by increasing incubation to +32° C., demonstrating linearity.

TABLE 2
Relative growth rate matrix. Relative growth rate (%) was
determined by dividing the average growth rate of each culture
by either wildtype or empty vector containing yeast.

Average Growth rate (%)

| | vs Wildtype | vs Empty Vector |
|---|---|---|
| Wildtype | 100.0% | — |
| Empty Vector | 65.4% | 100.0% |
| EVA @ +30° C. | 38.1% | 58.2% |
| EVA @ +32° C. | 14.2% | 21.7% |

### Example 4: Rescue of EVA Growth Reduction by an Augmented Mitochondrial Antioxidant System

### Co-Expression of Chimera and EVA Significantly Rescues EVA-Dependent Growth Rate Reduction

As a potent antioxidant and key modulator of metabolism via lipoylation of both PDH and aKDH enzymes, it was hypothesised that overproduction of lipoic acid may convey an increased resistance to oxidative stress within the cell. Cellular growth rate was used as a measure of cellular fitness under a variety of oxidative conditions, FIG. 5.

As demonstrated above, expression of the tick polypeptide analogue, EVA, resulted in a severe (approximately 60% relative to wildtype and 40% relative to control) decrease in cellular fitness (growth rate). EVA and the Chimeric protein were co-expressed with the hypothesis that an augmented antioxidant system may serve to rescue the observed phenotypes.

Table 3 details the experimental design of growth rate rescue experiments. In total, the growth rates of four independent yeast strains were compared. A ‘control’ strain which carried the ‘empty’ plasmid was used to determine the background metabolic effect of maintaining a low-copy (centromeric) plasmid with no recombinant protein being expressed. All yeast were of the same genetic background.

TABLE 3
Experimental design of growth rate rescue experiments

| Strain | ‘Empty’ plasmid | +Chimera | +EVA |
|---|---|---|---|
| Control | Y | N | N |
| Chimera | N | Y | N |
| EVA | N | N | Y |
| Chimera; +EVA | N | Y | Y |

Abbreviations: Y = Yes, N = No, e.g., Chimera; +EVA strain expresses both Chimeric and EVA.

Data for these experiments are summarised in FIG. 9 and Table 4.

TABLE 4
Summary table of FIG. 9. Mean average growth rates, standard
deviation, and coefficient of variation for each strain,

| | Growth | Standard | Coefficient of |
|---|---|---|---|
| Strain | Rate, μ | Deviation | Variation (%) |
| Empty Vector | 0.446 | ±0.0435 | 9.8% |
| Chimera | 0.483 | ±0.0496 | 10.3% |
| EVA | 0.259 | ±0.0378 | 14.6% |
| Chimera; +EVA | 0.451 | ±0.0674 | 14.9% |

Data in FIG. 9 and Table 4 also demonstrated that when rescued, the growth of Chimera; +EVA cultures were statistically insignificant from those of control (+empty vector, or non-EVA expressing) cultures, suggesting that expression of the chimera was sufficient alone to store cellular fitness.

These experiments also demonstrated that chimera expression reversed the temperature-dependent loss of growth rate in the EVA-expressing yeast.

Coexpression of Chimeric Fusion with Two Other ICK-Peptides, Purotoxin-1 and Psalmotoxin-1 Elicits a Similar Response as with EVA

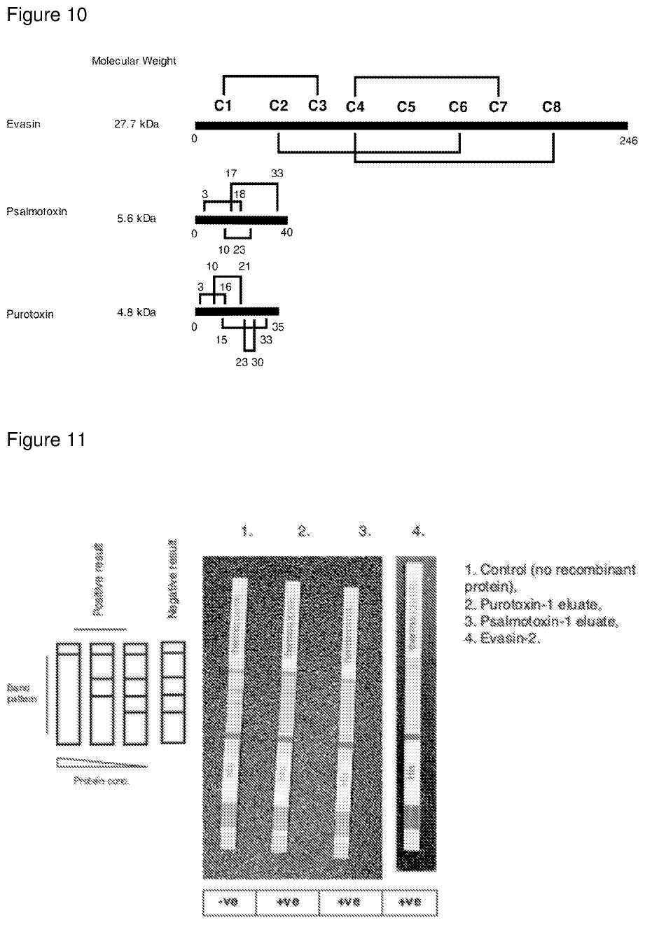

As the data demonstrated that the chimeric strain could significantly rescue the growth of one ICK polypeptide, it was investigated whether two more ICK-expressing strains could also be rescued. Expression constructs for the venom peptides, ‘Purotoxin’ (UniProt ID: TXPR1_ALOMR) from the Wolf Spider, Alopecosa marikovskyi (see SEQ ID NOs: 20 and 21) and ‘Psalmotoxin-1’ (UniProt ID: TXP1_PSACA) from the Trinidad chevron tarantula Psalmopoeus cambridgei (see SEQ ID NOs: 22 and 23) were transformed into yeast cultures and their growth rates monitored as above. Both peptides have well-described therapeutic potential as either analgesia or antimalarial agents and in conjunction with other ICK peptides contain 4 and 3 disulfide bonds, respectively. A schematic of each peptide is given in FIG. 10. Expression of both polypeptides was confirmed by an antibody-based assay (FIG. 11).

In total, the growth rates of four independent yeast strains were compared (see Table 5). A ‘control’ strain which carried the ‘empty’ plasmid was used to determine the background metabolic effect of maintaining a low-copy (centromeric) plasmid with no recombinant protein being expressed. All yeast were of the same BY4741

### BACKGROUND

TABLE 5
Experimental design of growth rate rescue experiments

| | ‘Empty’ | | +Purotoxin | +Psalmotoxin |
|---|---|---|---|---|
| Strain | plasmid | +Chimera | (PUR) | (PSA) |
| Control | Y | N | N | N |
| Chimera | N | Y | N | N |
| PURO-1 | N | N | Y | N |
| Chimera + PURO-1 | N | Y | Y | N |
| PSA | N | N | N | Y |
| Chimera + PSA | N | Y | N | Y |

Abbreviations: Y = Yes, N = No.

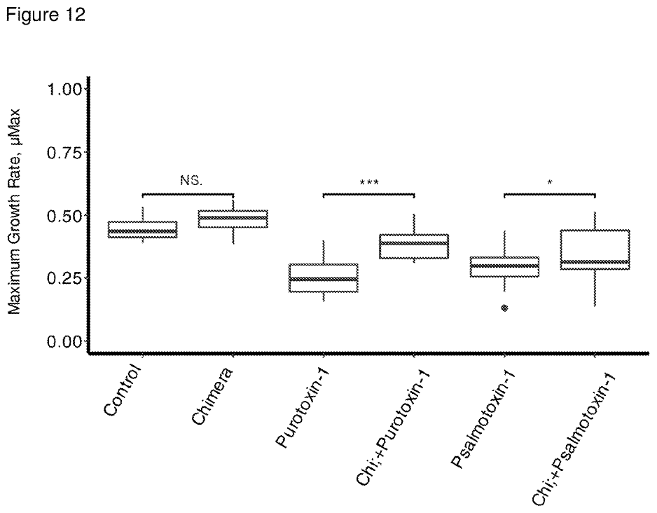

TABLE 6
Summary table of FIG. 12 Mean average growth rates, standard
deviation and coefficient of variation for each strain

| | Average | Standard | Coefficient of |
|---|---|---|---|
| Strain | Growth Rate, μ | Deviation | Variation (%) |
| Empty Vector | 0.446 | +0.0435 | 9.8% |
| Chimera | 0.483 | +0.0496 | 10.3% |
| PURO-1 | 0.258 | +0.073 | 28.4% |
| Chimera + PURO-1 | 0.390 | +0.060 | 15.5% |
| PSA | 0.298 | +0.061 | 20.5% |
| Chimera + PSA | 0.355 | +0.103 | 28.9% |

The data shown in Table 6 above (also given graphically in FIG. 12) demonstrates that again co-expression of the Chimeric fusion was sufficient to restore the growth rates of cells expressing either of the ICK peptides. Compared to the expression of the Evasin (EVA), more variability within the data sets were demonstrated. This is particularly true with regards to the growth of the Psalmotoxin-1 expressing yeast (Psalmotoxin-1), possibly reflecting a tolerance towards the relatively less complex (in terms of disulfides) polypeptide.

When viewed in combination (FIGS. 10 and 12), the number of disulfides (S—S) present in each venom peptide (Table 6) appears to infer how well (or not) the yeast cultures will respond (growth rate) to their expression. This effect appears to be independent of the molecular weight of each peptide (Table 5). Such that a high number of disulfides (Evasin and Purotoxin, 4) resulted in a greater reduction in growth rate versus a lower number (Psalmotoxin, 3). Likewise, the Chimeric-dependent growth rate rescue also reflects this finding, with a stronger rescue in the higher molecular weight (26 kDa) and higher number of disulfides (4), FIG. 10 and Table 6.

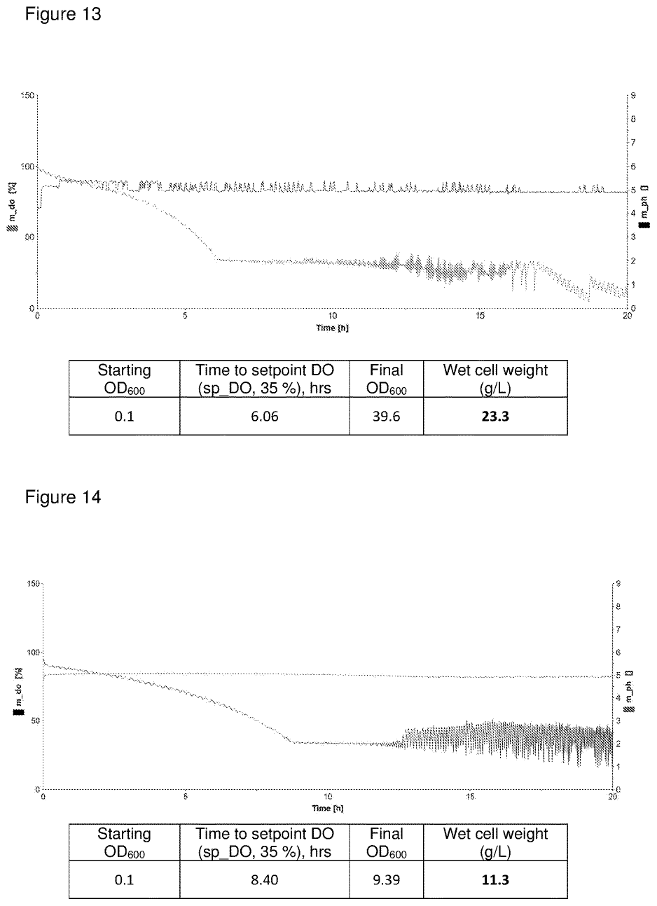

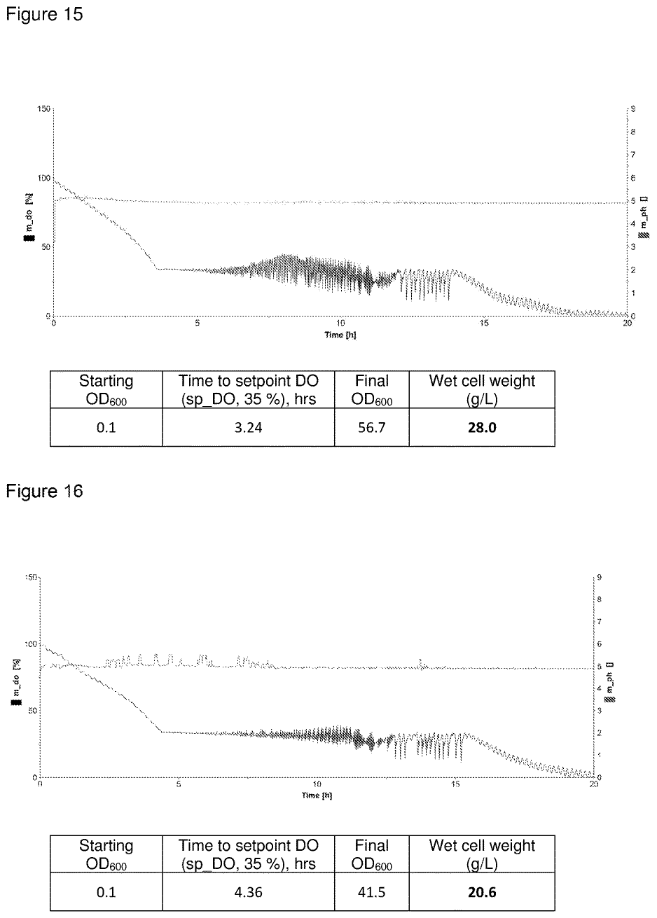

Next, we performed pilot-scale 100 mL fermentations in order to identify whether the above growth rate observations (namely, Purotoxin and Evasin) could transfer to a commercially relevant batch fermentation system. These data are presented in FIGS. 13 through 16. In four separate batch fermentations, expression of the chimera conferred faster growth metrics (time to set point DO), as well as final yield in terms of both culture (optical) density (OD₆₀₀) and wet cell weight. Of particular note, these batch fermentations aligned with growth measurements taken in FIG. 9 and FIG. 12, namely that the high-molecular-weight Evasin is better supported than purotoxin-1 within the yeast system by chimera-expression.

Recombinant expression of a disulfide-rich ICK infers heightened flux through the oxidative protein folding pathway and as a result, increased production of radical species. Given that each disulfide bond forms a stoichiometric quantity of radical oxygen species via Ero1-dependent oxidation of cysteine thiols (Tyo et al., 2012, BMC Biology, 10.), we hypothesised that heterologous expression of an ICK peptide likely also results in ‘heightened’ production of oxidants, placing great strain upon the protein folding machinery (including the UPR), resulting in dire consequences for growth rates and final product yields. This is demonstrated by the differences in growth rates of Evasin, Purotoxin-1 and Psalmotoxin, wherein the peptide with the lowest number of disulfides (Psalmotoxin, disulfides n=3) appeared to be better tolerated (less impact on growth rate) than either Evasin or Purotoxin-1 (each with 4 disulfides).

We were able to demonstrate that by co-expressing an altered version of a key antioxidant pathway via chimera, ICK-expressing yeast no longer exhibited slow growth rates and their fitness appeared to be restored. We suggest that this was caused by an indirect antioxidant ‘buffering’ effect levied by the expression of the chimeric protein. This would have the effect of allowing the yeast host to better tolerate the folding ‘cost’ of recombinant ICKs by preventing radicals from damaging key biomolecules (nucleic acids, lipids, proteins, etc.) and compromising the fitness of the cell.

1. A chimeric protein which comprises a Bol3 polypeptide operably linked to a Lip5 polypeptide.

2. The chimeric protein as claimed in claim 1 wherein said Bol3 polypeptide has at least 50% sequence to the amino acid sequence of SEQ ID NO: 1.

3. The chimeric protein as claimed in claim 1 wherein said Lip5 polypeptide has at least 50% sequence to the amino acid sequence of SEQ ID NO: 2.

4. The chimeric protein as claimed in claim 1, wherein said chimeric protein includes a linker sequence between the Bol3 polypeptide and the Lip5 polypeptide.

5. (canceled)

6. (canceled)

7. The chimeric protein as claimed in claim 1, wherein said protein has at least 70% sequence identity to the amino acid sequence of SEQ ID NO: 3.

8. The chimeric protein as claimed in claim 4 wherein said linker sequence comprises a Cia2 sequence.

9. The chimeric protein as claimed in claim 8, wherein the linker sequence is selected from the amino acid sequence of SEQ ID No: 28 and SEQ ID No: 29.

10. (canceled)

11. The chimeric protein as claimed in any one of the claim 8, wherein said chimeric protein has at least 70% sequence identity to the amino acid sequence of SEQ ID NO: 31.

12. The chimeric protein as claimed in claim 1 which is expressed from the polynucleotide of SEQ ID No: 32.

13. A polynucleotide which encodes the chimeric protein as claimed in claim 1.

14. The polynucleotide as claimed in claim 13 which comprises a sequence having at least 50% sequence identity to the nucleotide sequence of SEQ ID NO: 4 and/or a sequence having at least 50% sequence identity to the nucleotide sequence of SEQ ID NO: 5.

15. The polynucleotide as claimed in claim 13 which encodes a polypeptide having a sequence with at least 90% sequence identity to the nucleotide sequence of SEQ ID NO: 3.

16. The polynucleotide as claimed in claim 13 which has at least 70% sequence identity to SEQ ID NO: 32.

17. (canceled)

18. (canceled)

19. (canceled)

20. An expression system for expressing a target polypeptide in a host cell, said system comprising an expression vector comprising a polynucleotide as claimed in claim 13, said vector also including a cloning site for insertion of a polynucleotide encoding a target polypeptide.

21. An expression system for expressing a target polypeptide in a host cell, said system comprising an expression vector comprising a polynucleotide as claimed in claim 13 and an expression vector encoding the target polypeptide.

22. The expression system as claimed in claim 21, wherein said target polypeptide has at least one disulfide bond.

23. (canceled)

24. The expression system as claimed in claim 22 wherein the target polypeptide comprises 2 to 5 disulfide bonds and the disulfide bonds of said target polypeptide form an ICK.

25. A method for producing a target polypeptide having at least one disulfide bond within a host cell, wherein said method includes: culturing a host cell transformed with a polynucleotide as claimed in claim 13 under conditions suitable for the expression of the chimeric protein together with expression of the target polypeptide.

26. (canceled)

27. (canceled)

28. The method as claimed in claim 25 wherein the target polypeptide comprises 2 to 5 disulfide bonds and the disulfide bonds of said target polypeptide form an ICK.

29. The method as claimed in claim 25 wherein said target peptide is a venom peptide.

30. The method as claimed in claim 25 wherein said host cell is genetically engineered to express said target polypeptide.


SEQUENCE LISTING (32 sequences)

SEQ ID NO: 1 - protein, 118 aa, Saccharomyces cerevisiae
  REGION 1..118: note = Bol3
MKLPQTMLRSISVKHVRWPRILTGSKLWYSTQMAMTPEEKMITDKLQQELEPEVCKVQDVSGGCGSMFAINITSKKFNGLSLIKQHQLVNRILRDDISRWHGLQLTTKKSTGKGPASS

SEQ ID NO: 2 - protein, 414 aa, Saccharomyces cerevisiae
  REGION 1..414: note = Lip5
MYRRSVGVLFVGRNTRWISSTIRCGTSATRPIRSNALNTDSDNASVRVPVGNSTEVENATSQLTGTSGKRRKGNRKRITEFKDALNLGPSFADFVSGKASKMILDPLEKARQNTEEAKKLPRWLKVPIPKGTNYHKLKGDVKELGLSTVCEEARCPNIGECWGGKDKSKATATIMLLGDTCTRGCRFCSVKTNRTPSKPDPMEPENTAEAIKRWGLGYVVLTTVDRDDLVDGGANHLAETVRKIKQKAPNTLVETLSGDFRGDLKMVDIMAQCGLDVYAHNLETVESLTPHVRDRRATYRQSLSVLERAKATVPSLITKTSIMLGLGETDEQITQTLKDLRNIQCDVVTFGQYMRPTKRHMKVVEYVKPEKFDYWKERALEMGFLYCASGPLVRSSYKAGEAFIENVLKKRNMK

SEQ ID NO: 3 - protein, 541 aa, synthetic construct
  REGION 1..541: note = Chimeric Protein Bol3-Lip5 with His Linker
MKLPQTMLRSISVKHVRWPRILTGSKLWYSTQMAMTPEEKMITDKLQQELEPEVCKVQDVSGGCGSMFAINITSKKFNGLSLIKQHQLVNRILRDDISRWHGLQLTTKKSTGKGPASSHHHHHHHHHHYRRSVGVLFVGRNTRWISSTIRCGTSATRPIRSNALNTDSDNASVRVPVGNSTEVENATSQLTGTSGKRRKGNRKRITEFKDALNLGPSFADFVSGKASKMILDPLEKARQNTEEAKKLPRWLKVPIPKGTNYHKLKGDVKELGLSTVCEEARCPNIGECWGGKDKSKATATIMLLGDTCTRGCRFCSVKTNRTPSKPDPMEPENTAEAIKRWGLGYVVLTTVDRDDLVDGGANHLAETVRKIKQKAPNTLVETLSGDFRGDLKMVDIMAQCGLDVYAHNLETVESLTPHVRDRRATYRQSLSVLERAKATVPSLITKTSIMLGLGETDEQITQTLKDLRNIQCDVVTFGQYMRPTKRHMKVVEYVKPEKFDYWKERALEMGFLYCASGPLVRSSYKAGEAFIENVLKKRNMK

SEQ ID NO: 4 - DNA, 354 bp, Saccharomyces cerevisiae
  misc_feature 1..354: note = Bol3 minus native stop codon
  source 1..354: mol_type = genomic DNA
atgaagctcccacagaccatgctacgttctatatctgtgaagcatgtccggtggccaaggattctgacgggctcaaagctttggtactcaacgcagatggcaatgactccggaggagaagatgatcaccgataaactacaacaggaactggaacctgaagtgtgtaaagtgcaagacgtttccggtggctgcggatccatgtttgctatcaacataacaagcaagaagttcaacggactgagtctcatcaagcagcaccagctggtgaacagaattttgagggacgatatttccagatggcatggcctacaattgaccactaagaagtcaactgggaagggtccggcatcatca

SEQ ID NO: 5 - DNA, 1242 bp, Saccharomyces cerevisiae
  misc_feature 1..1242: note = Lip5 minus native start codon
  source 1..1242: mol_type = genomic DNA
tatagacgatctgttggagtactatttgttgggagaaatacaagatggatttcgtccactattaggtgtggaacgagcgcaactcggccaataagatccaatgcattgaatactgattcagataacgctagcgttagggtaccagttggaaattcaactgaggttgaaaatgcaaccagtcagttaacagggacttcgggaaaaagacgcaaggggaacagaaaaagaattacagagttcaaagatgctcttaatttgggtccatcttttgcagattttgtttcaggtaaagcttcgaaaatgatcttggaccctctggagaaggcaaggcaaaatacagaggaagctaaaaaactacctcgttggcttaaggttcctattcctaagggtaccaattaccataaattgaaaggcgacgtaaaagaattaggcctgagtactgtttgtgaggaggcaagatgtcccaatattggtgaatgttggggaggcaaagataaatctaaggcaacggcaacaattatgctgcttggtgatacttgcactcgtggatgtaggttttgttctgtgaagaccaatagaacgcctagtaagccggacccaatggagcccgaaaatactgccgaagctatcaaaagatgggggttgggttatgttgttttaactaccgttgatagggacgatttagtcgatggtggtgctaatcacctggccgagacggttcgcaaaatcaaacagaaggcaccaaatactcttgtagagactctttctggtgatttcagaggtgatttgaagatggtggacattatggcacaatgtgggcttgatgtttacgcacataatttggaaacagttgaatcactaacaccacatgtcagagacagaagagctacttatagacagtctttgagtgttttagaaagggcaaaagctacggttccgtcactgattactaaaacatcaataatgctgggtctaggagaaactgatgagcaaattactcaaactttgaaggatttgcgcaatattcaatgtgatgttgttacatttggtcaatatatgaggccaaccaagagacatatgaaagtcgtagaatatgtgaaacccgaaaagtttgactactggaaagagagagctttagagatgggattcttgtattgtgcatctggacccctagtaagatcgtcatataaggctggtgaagcatttattgagaacgttttgaagaaaagaaacatgaaataa

SEQ ID NO: 6 - protein, 117 aa, Ixodes ricinus
  REGION 1..117: note = Evasin-P1096_ir
MELNAFTILHIAVFIAVGYYANTHTTVTGSVEGKPNNPNEDIEVSYCRMNCTVENGVSSACSGDCVCVHRDNEPNGICVEITYFGDFGDPSQDPSIDEAAPRESVSKRRSNGESKTP

SEQ ID NO: 7 - protein, 242 aa, Amblyomma cajennense
  REGION 1..242: note = Evasin_ac2
MHLAALLTCLIVFFGSAACQDLTVEDSITGTDLNITDDIARPLPPEVLPINGTEQVGNSTRNGTSTDAITTTSATTTTTNISSTAEATSASTTRTKRTRRPRPTKKTKRPHPKEDDKYGTLIDSKGCEHKVLQSKHDLYTATCTGTCRGRTYPIVDGTPCLHAVRRSPRLNKGGKKCLKGVCRRGRCQRPFKKEVKCKVPKGTVHYYDDNGENYSYDDENYGTYYDYYSSYENVDSNDRLAE

SEQ ID NO: 8 - protein, 263 aa, Amblyomma cajennense
  REGION 1..263: note = Evasin_ac1
MHLAALLTCLLVFFGSAACQDLTEEDSTALIDVNGTDDNAHPLPPDFLPINGTEQVGNSTGDEGTEASTPEEPIVLGIITEGGPRPDATTTTSATTTTVNISSTAEATSASTTRTKRTRRPRPTKKTKRPHPKEDEKYGTLIDSKGCEHKVLQSKHDLYTATCTGTCRGRTYPIVDGTPCLHAVRRSPRLNKGGKKCLKGVCRRGRCQLPFKKEVKCQVPKGTVHYYDDNGGDYNYNDDINHGTYYDYDNSYEGVNYNHRFAE

SEQ ID NO: 9 - protein, 127 aa, Rhipicephalus sanguineus
  REGION 1..127: note = Evasin-4_rs
MAFKYWFVFAAVLYARQWLSTKCEVPQMTSSSAPDLEEEDDYTAYAPLTCYFTNSTLGLLAPPNCSVLCNSTTTWFNETSPNNASCLLTVDFLTQDAILQENQPYNCSVGHCDNGTCAGPPRHAQCW

SEQ ID NO: 10 - protein, 120 aa, Amblyomma americanum
  REGION 1..120: note = Evasin-P1243_aa
MHSTIAYVFVSALALFAALHGSTSARNHTEDNSTEYYDYEEARCACPARHLNNTNGTVLKLLGCHYFCNGTLCTAPDGYPCYNLTAQQVRTLTTYPNTSCAVGVCMKGTCVKNGTMEQCF

SEQ ID NO: 11 - protein, 114 aa, Rhipicephalus sanguineus
  REGION 1..114: note = Evasin-1_rs
MTFKACIAIITALCAMQVICEDDEDYGDLGGCPFLVAENKTGYPTIVACKQDCNGTTETAPNGTRCFSIGDEGLRRMTANLPYDCPLGQCSNGDCIPKETYEVCYRRNWRDKKN

SEQ ID NO: 12 - protein, 115 aa, Amblyomma maculatum
  REGION 1..115: note = Evasin-P1182_am
MALNWSFRVIFVSTMWCALLKFATLGEPKDDNDYGGGCPFVVLGNGTHAKPAGCSHLCNGAPETLDNIECYNVTEEVAKRMTPGIPYACWLGWCNKGECKRGNRTEVCYRGSEEE

SEQ ID NO: 13 - protein, 115 aa, Amblyomma triste
  REGION 1..115: note = Evasin-P1183_at
MTRNWSFRVIFVSAMWCALLKFATLEAPKDDFEYDGGCPFVVLDNGTHVKPAGCSHLCNGAPETLDNIECYNVTEEVAKRMTPGIPYACWLGWCSKGECKRDNRTEVCYRGSEEE

SEQ ID NO: 14 - DNA, 80 bp, synthetic construct
  misc_feature 1..80: note = Lip5 Forward Primer
  source 1..80: mol_type = other DNA
caccatcaccatcaccatcaccatcaccatcaccttaggctttatagacgatctgttggagtactatttgttgggagaaa

SEQ ID NO: 15 - DNA, 61 bp, synthetic construct
  misc_feature 1..61: note = Lip5 Reverse Primer
  source 1..61: mol_type = other DNA
cccccccgcggttattatttcatgtttcttttcttcaaaacgttctcaataaatgcttcac

SEQ ID NO: 16 - DNA, 46 bp, synthetic construct
  misc_feature 1..46: note = Bol3 Forward Primer
  source 1..46: mol_type = other DNA
ccccctctagatacacaatgaagctcccacagaccatgctacgttc

SEQ ID NO: 17 - DNA, 68 bp, synthetic construct
  misc_feature 1..68: note = Bol3 Reverse Primer
  source 1..68: mol_type = other DNA
atggtgatggtgatggtgatggtgatggtgatgaggcctaagtgatgatgccggacccttcccagttg

SEQ ID NO: 18 - protein, 248 aa, synthetic construct
  REGION 1..248: note = Evasin (EVA)
MHHHHHHHLAALLTCLIVFFGSAACQDLTVEDSITGTDLNITDDIARPLPPEVLPINGTEQVGNSTRNGTSTDAITTTSATTTTTNISSTAEATSASTTRTKRTRRPRPTKKTKRPHPKEDDKYGTLIDSKGCEHKVLQSKHDLYTATCTGTCRGRTYPIVDGTPCLHAVRRSPRLNKGGKKCLKGVCRRGRCQRPFKKEVKCKVPKGTVHYYDDNGENYSYDDENYGTYYDYYSSYENVDSNDRLAE

SEQ ID NO: 19 - DNA, 744 bp, synthetic construct
  misc_feature 1..744: note = Evasin (EVA)
  source 1..744: mol_type = other DNA
atgcatcaccatcaccatcaccacttagcggctctgcttacgtgtctaattgtttttttcggttctgccgcctgccaagatttgacagtggaagactctataactggaactgatctaaacataacagatgatatagcaagaccattaccgcctgaagttctacctataaatggaactgagcaagtggggaattctacccgtaatggtactagcacagacgctataacaacaacatcagcgactacaacaacaacaaacatttcaagtaccgctgaagctacatctgcttccacaacaagaaccaagaggaccagaaggcctcgtcccaccaaaaaaactaaacgtccacatccaaaagaagatgataagtacggaacgctaattgattcaaaaggctgtgaacacaaggtattgcaatctaaacacgacctttacacggctacctgcacaggcacatgtagaggtagaacatatcctatagtcgatggcactccatgtttgcatgctgttagacgtagtccaagattgaacaaaggaggtaaaaagtgtttaaaaggtgtttgcagaagagggaggtgtcaacgtcctttcaaaaaagaagtcaaatgcaaagttccgaaaggcaccgtgcattattatgatgataacggtgaaaattattcatatgatgacgagaattacggcacctattatgactattattcatcttacgagaatgttgattcaaatgacagattagctgaa

SEQ ID NO: 20 - protein, 35 aa, Alopecosa marikovskyi
  REGION 1..35: note = Purotoxin 1
GYCAEKGIRCDDIHCCTGLKCKCNASGYNCVCRKK

SEQ ID NO: 21 - DNA, 108 bp, Alopecosa marikovskyi
  misc_feature 1..108: note = Purotoxin-1
  source 1..108: mol_type = genomic DNA
atgggttattgtgctgaaaaaggtattagatgtgatgatattcattgttgtactggtttgaaatgtaaatgtaatgcttctggttataattgtgtttgtagaaaaaaa

SEQ ID NO: 22 - protein, 40 aa, Psalmopoeus cambridgei
  REGION 1..40: note = Psalmotoxin
EDCIPKWKGCVNRHGDCCEGLECWKRRRSFEVCVPKTPKT

SEQ ID NO: 23 - DNA, 123 bp, Psalmopoeus cambridgei
  misc_feature 1..123: note = Psalmotoxin
  source 1..123: mol_type = genomic DNA
atggaagattgtattccaaaatggaaaggttgtgttaatagacatggtgattgttgtgaaggtttggaatgttggaaaagaagaagatcttttgaagtttgtgttccaaaaactccaaaaact

SEQ ID NO: 24 - DNA, 37 bp, synthetic construct
  source 1..37: mol_type = other DNA
ccccctctagatacacaatgaagctcccacagaccat

SEQ ID NO: 25 - DNA, 41 bp, synthetic construct
  source 1..41: mol_type = other DNA
ggggggcggccgcaggcctaagtgatgatgccggacccttc

SEQ ID NO: 26 - DNA, 91 bp, synthetic construct
  source 1..91: mol_type = other DNA
cccccgcggccgcttctggggagcggcctgtgacggcaggcgaggaggaccttaggctttatagacgatctgttggagtactatttgttgg

SEQ ID NO: 27 - DNA, 56 bp, synthetic construct
  source 1..56: mol_type = other DNA
gggggccgcggttattatttcatgtttcttttcttcaaaacgttctcaataaatgc

SEQ ID NO: 28 - protein, 12 aa, Saccharomyces cerevisiae
SGERPVTAGEED

SEQ ID NO: 29 - protein, 21 aa, synthetic construct
LRPAAASGERPVTAGEEDLRL

SEQ ID NO: 30 - DNA, 63 bp, synthetic construct
  source 1..63: mol_type = other DNA
cttaggcctgcggccgcttctggggagcggcctgtgacggcaggcgaggaggaccttaggctt

SEQ ID NO: 31 - protein, 552 aa, synthetic construct
MKLPQTMLRSISVKHVRWPRILTGSKLWYSTQMAMTPEEKMITDKLQQELEPEVCKVQDVSGGCGSMFAINITSKKFNGLSLIKQHQLVNRILRDDISRWHGLQLTTKKSTGKGPASSLRPAAASGERPVTAGEEDLRLYRRSVGVLFVGRNTRWISSTIRCGTSATRPIRSNALNTDSDNASVRVPVGNSTEVENATSQLTGTSGKRRKGNRKRITEFKDALNLGPSFADFVSGKASKMILDPLEKARQNTEEAKKLPRWLKVPIPKGTNYHKLKGDVKELGLSTVCEEARCPNIGECWGGKDKSKATATIMLLGDTCTRGCRFCSVKTNRTPSKPDPMEPENTAEAIKRWGLGYVVLTTVDRDDLVDGGANHLAETVRKIKQKAPNTLVETLSGDFRGDLKMVDIMAQCGLDVYAHNLETVESLTPHVRDRRATYRQSLSVLERAKATVPSLITKTSIMLGLGETDEQITQTLKDLRNIQCDVVTFGQYMRPTKRHMKVVEYVKPEKFDYWKERALEMGFLYCASGPLVRSSYKAGEAFIENVLKKRNMK

SEQ ID NO: 32 - DNA, 1662 bp, synthetic construct
  source 1..1662: mol_type = other DNA
atgaagctcccacagaccatgctacgttctatatctgtgaagcatgtccggtggccaaggattctgacgggctcaaagctttggtactcaacgcagatggcaatgactccggaggagaagatgatcaccgataaactacaacaggaactggaacctgaagtgtgtaaagtgcaagacgtttccggtggctgcggatccatgtttgctatcaacataacaagcaagaagttcaacggactgagtctcatcaagcagcaccagctggtgaacagaattttgagggacgatatttccagatggcatggcctacaattgaccactaagaagtcaactgggaagggtccggcatcatcacttaggcctgcggccgcttctggggagcggcctgtgacggcaggcgaggaggaccttaggctttatagacgatctgttggagtactatttgttgggagaaatacaagatggatttcgtccactattaggtgtggaacgagcgcaactcggccaataagatccaatgcattgaatactgattcagataacgctagcgttagggtaccagttggaaattcaactgaggttgaaaatgcaaccagtcagttaacagggacttcgggaaaaagacgcaaggggaacagaaaaagaattacagagttcaaagatgctcttaatttgggtccatcttttgcagattttgtttcaggtaaagcttcgaaaatgatcttggaccctctggagaaggcaaggcaaaatacagaggaagctaaaaaactacctcgttggcttaaggttcctattcctaagggtaccaattaccataaattgaaaggcgacgtaaaagaattaggcctgagtactgtttgtgaggaggcaagatgtcccaatattggtgaatgttggggaggcaaagataaatctaaggcaacggcaacaattatgctgcttggtgatacttgcactcgtggatgtaggttttgttctgtgaagaccaatagaacgcctagtaagccggacccaatggagcccgaaaatactgccgaagctatcaaaagatgggggttgggttatgttgttttaactaccgttgatagggacgatttagtcgatggtggtgctaatcacctggccgagacggttcgcaaaatcaaacagaaggcaccaaatactcttgtagagactctttctggtgatttcagaggtgatttgaagatggtggacattatggcacaatgtgggcttgatgtttacgcacataatttggaaacagttgaatcactaacaccacatgtcagagacagaagagctacttatagacagtctttgagtgttttagaaagggcaaaagctacggttccgtcactgattactaaaacatcaataatgctgggtctaggagaaactgatgagcaaattactcaaactttgaaggatttgcgcaatattcaatgtgatgttgttacatttggtcaatatatgaggccaaccaagagacatatgaaagtcgtagaatatgtgaaacccgaaaagtttgactactggaaagagagagctttagagatgggattcttgtattgtgcatctggacccctagtaagatcgtcatataaggctggtgaagcatttattgagaacgttttgaagaaaagaaacatgaaataataa